(19) United States
(12) Patent Application Publication
(10) Pub. No.: US 2012/0239086 A1
(43) Pub. Date: Sep. 20, 2012

(54) NONLINEAR SELF SEATING SUTURE ANCHOR FOR CONFINED SPACES

(76) Inventors: Alan M. Reznik, Woodbridge, CT (US); Raymond A. Sachs, San Diego, CA (US)

(21) Appl. No.: 13/418,817

(22) Filed: Mar. 13, 2012

Related U.S. Application Data
(60) Provisional application No. 61/452,453, filed on Mar. 14, 2011.
(60) Provisional application No. 61/473,389, filed on Apr. 8, 2011.
(60) Provisional application No. 61/570,985, filed on Dec. 15, 2011.
(60) Provisional application No. 61/594,606, filed on Feb. 3, 2012.

(51) Int. Cl. A61B 17/04

(52) U.S. Cl. 606/232

(57) ABSTRACT

A self-seating, non-linear suture anchor system and method of insertion is disclosed where the anchor is made up of a non-linear or curved anchor. The anchor has at least one hole for one or more sutures such that the anchor can be introduce through a small opening, incision, channel or cannula in a confined space and inserted at an angle different than, or divergent from, the angle determined by the cannula, opening or the confines of the space.

### CROSS REFERENCE TO RELATED APPLICATION

This application claims the benefit of U.S. Provisional Application Nos. 61/452,453, entitled “NONLINEAR SELF SEATING SUTURE ANCHOR FOR CONFINED SPACES”, filed Mar. 14, 2011, 61/473,389, entitled “NONLINEAR SELF SEATING SUTURE ANCHOR FOR CONFINED SPACES”, filed Apr. 8, 2011, 61/570,985, “NONLINEAR SELF SEATING SUTURE ANCHOR FOR CONFINED SPACES”, filed Dec. 15, 2011, and 61/594,606, entitled “NONLINEAR SELF SEATING SUTURE ANCHOR FOR CONFINED SPACES”, filed Feb. 3, 2012.

### BACKGROUND OF THE INVENTION

1. Field of the Invention

The present invention relates to a suture anchor, a device for inserting the suture anchor, and a method for attaching suture materials to body tissue, such as bone or cartilage to repair injuries to tendons, ligaments, cartilage and other connecting structures in the body.

2. Description of the Related Art

Suture anchors are an effective way to attach soft tissues to bone or cartilage. There are a large number of anchors, anchor systems and insertion devices in the literature and there are many technologies related to anchors and fastening systems for this purpose. These technologies primarily focus upon creating secure fixation and improving pull out strength. These technologies have also been developed to accommodate single or multiple sutures, non-locking, locking and self-locking designs, as well as those that require knots and knotless designs. Such devices have become the mainstay of open and endoscopic surgical repairs in many parts of the body.

### SUMMARY OF THE INVENTION

The present suture anchor is designed with a curved body and beveled tip to facilitate to engagement with a tunnel (or hole) formed in a bone or cartilage support surface that is not aligned with the insertion cannula (that is, guide or insertion device) or control rod. As such, the present invention provides for the non-linear application of suture anchors. The invention represents a paradigm shift from the current state of the art for inserting anchors in a confined space in the body. The invention teaches a novel approach by applying a curved anchor through a guide (or inserter) providing for lateral deployment and thereby avoiding the many limitations and difficulties created by the current state of the art of applying anchors of any type with straight or curved inserters. These difficulties include limits on the angle of insertion, the size of the guide, and the cannulas being used; all of which are overcome by the present invention.

The anchor itself has one or more holes for suture material. The anchor also has a textured, ribbed or barbed surface to gain purchase on the walls of the tunnel and improve fixation. The anchor is attached to a control rod that has flexibility in its design to allow the anchor to redirect itself into the bone tunnel.

The control rod (or applicator) may be made of a flexible or malleable material such as a spring, spring like materials or shape memory material such as Nitinol. It may be constructed with a hinged attachment site and a more rigid control rod with an angled entry in the mounting section of the anchor to allow for non-linear insertion of the anchor. This angled section can be constructed to control the minimum and maximum deflection of the anchor from the direction of the control rod to aid in control upon deployment of the anchor. The control rod can be made with a hinge or flexible section to allow for non-linear insertion of the anchor. The control rod and anchor are accompanied by a matching guide and flexible awl or reamer capable of creating a tunnel at the desired angle even when that angle is different from the insertion angle or direction of the cannula, guide and reamer. The invention of a non-linear anchor, adaption of the control rod/anchor junction, a flexible or hinged control rod, angled guides and flexible reamer constitute a unique system and method of non-linear or divergent application of a suture anchor that allows for surgical repairs in anatomically confined spaces. Alternatively the non-linear anchor may be constructed with a cannulation hole in the body or the anchor in the direction of its application to be inserted over a curved or flexible guide wire. This cannulated non-linear anchor can be located by first inserting the guide wire, sliding the device down the wire and then pushing it over the wire in place.

With the foregoing in mind, it is, therefore, an object of the present invention to provide a suture anchor and delivery assembly including an elongated, non-linear anchor body with a first end and a second end. At least one hole is formed in the anchor body for the passage of a suture therethrough, the anchor body including an attachment site at a second end thereof. A control rod shaped and dimensioned for attachment to the attachment site of the anchor body is provided. The control rod includes a distal end constructed of a flexible materials allowing for displacement of the control rod. A guide for the insertion of the anchor is also provided.

It is also an object of the present invention to provide a suture anchor and delivery assembly wherein the control rod has a section of a flexible material shaped to permit bending only in one desired plane of motion while limiting the total bend angle to allow for optimal insertion of the anchor.

It is another object of the present invention to provide a suture anchor and delivery assembly wherein the anchor and the control rod are coupled via a ball joint.

It is a further object of the present invention to provide a suture anchor and delivery assembly wherein the guide is an elongated hollow member having a first end and a second end with a conduit extending between the first end and the second end allowing for the passage of the anchor. The guide includes an outlet end at the second end of the guide wherein the conduit turns allowing for deployment of the anchor at the second end of the guide in a direction substantially transverse to the longitudinal axis of the guide.

It is also an object of the present invention to provide a suture anchor and delivery assembly wherein the anchor includes a cannulation down a center of the anchor body.

It is another object of the present invention to provide a suture anchor and delivery assembly wherein the anchor includes a first anchor section and a second anchor section, the first anchor section and the second anchor section being coupled with hinges permitting relative movement.

It is a further object of the present invention to provide a suture anchor and delivery assembly wherein the anchor is triangular in shape when viewed along a cross section taken perpendicular to a longitudinal axis of the anchor body, and the guide is also triangular in shape when view along a cross section perpendicular to a longitudinal axis thereof.

It is also an object of the present invention to provide a suture anchor and delivery assembly wherein the guide includes a top wall extending between first and second lateral side walls of the guide. The first and second lateral side walls of the guide extend from the top wall at an acute angle such that they meet at a bottom wall of the guide.

It is another object of the present invention to provide a suture anchor and delivery assembly wherein the control rod includes a substantially rigid straight section along a proximal end thereof and a flexible section at a distal end, the flexible section being weakened by removal of material or the formation of cuts in the flexible section of the control rod.

It is a further object of the present invention to provide a suture anchor and delivery assembly wherein the flexible section includes gaps allowing for control of material properties to obtain appropriate stiffness for application of the anchor, and the size of each gap is such as to control maximum flexion angle possible in the control rod.

It is also an object of the present invention to provide a suture anchor and delivery assembly wherein each of the gaps includes an outwardly tapered section leading to an outer surface of the flexible section when viewed along a plane symmetrically bisecting the control rod along a longitudinal axis thereof. The outwardly tapered section is defined by first and second opposed walls of the respective projections that, when viewed along the plane symmetrically bisecting the control rod along the longitudinal axis thereof, move closer together as they extend from a free end of the projections toward an upper surface of the control rod such that when the flexible section reaches a desired extent of its flexible motion. The first and second opposed walls come into contact preventing further movement of the flexible section of the control rod.

It is another object of the present invention to provide a suture anchor and delivery assembly wherein the flexible section is formed with notches formed along a length of the flexible section.

It is a further object of the present invention to provide a suture anchor and delivery assembly wherein each of the notches include an outwardly tapered section leading to an outer surface of the flexible section and an enlarged central recess spaced from the outer surface of the flexible section when view along a plane symmetrically bisecting the control rod along the longitudinal axis thereof.

It is also an object of the present invention to provide a suture anchor and delivery assembly wherein the control rod includes tension cable controlling flexing of the flexible section.

It is another object of the present invention to provide a suture anchor and delivery assembly wherein the first end of the tension cable is attached to the control rod adjacent a distal tip of the control rod and a second end of the tension cable is attached to the proximal end of the control rod.

It is a further object of the present invention to provide a suture anchor and delivery assembly wherein the second end of the tension cable is attached a stop, shaped and dimensioned to engage with a wall of the inserter.

It is also an object of the present invention to provide a suture anchor and delivery assembly wherein the second end of the tension cable is attached to a stop, functioning as a manual trigger controlling the bending of the control rod.

It is another object of the present invention to provide a method for attaching sutures to body tissue, such as bone or cartilage to repair injuries to tendons, ligaments, cartilage and other connecting structures in the body by application of a bone anchor in a non-linear, non-co-linear or a divergent angle to the insertion angle required to approach the anatomic structure as dictated by the confines of the local anatomy. The method includes forming a tunnel or hole within an anatomical site, positioning a guide adjacent the tunnel or hole, the guide including an angled outlet end having an opening transverse to the longitudinal axis of the guide, passing a bone anchor through the guide to, and out of the outlet end of the guide, positioning the anchor with the tunnel or hole, and seating and locking the anchor in the tunnel or hole.

It is a further object of the present invention to provide a method wherein the an inner surface of the guide at the outlet end is provided with a partial spherical shape shaped and dimensioned to direct the anchor to the outlet at the outlet end of the guide and into the tunnel or hole.

It is also an object of the present invention to provide a method wherein the anchor is coupled to a control rod which is composed of a proximal rigid core and distal flexible segment.

It is a further object of the present invention to provide a method wherein the anchor is wherein the anchor is non-linear.

It is also an object of the present invention to provide a method wherein the anchor is curved.

It is another object of the present invention to provide a suture anchor for use in non-linear, non-co-linear or divergent angle deployment. The suture anchor includes an anchor body having a first end and a second end. At least one hole is formed in the anchor body for the passage of a suture therethrough, the anchor body including an attachment site at a second end thereof. The anchor body is triangular in shape when viewed along a cross section taken perpendicular to a longitudinal axis of the anchor body.

It is a further object of the present invention to provide a suture anchor wherein the anchor includes a top surface extending from a first end to a second end of the anchor body. First and second lateral sides extend from the top surface at an acute angle such that they meet at a bottom surface of the anchor body.

It is also an object of the present invention to provide a suture anchor wherein the anchor includes a suture hole that extends through the anchor body from the first lateral side thereof to the second lateral side thereof at a position between the top surface and the bottom surface.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 discloses the anatomy of the shoulder showing the limits of access to the superior humeral head and the anterior gleno-humeral joint;

FIG. 2 discloses the anatomy of the knee showing the limits of access to the intercondylar notch and the patella femoral joint;

FIGS. 3(a)-3(d) are side views illustrating four alternate embodiments of the non-linear suture anchor for application in a confined space;

FIGS. 4(a)-4(c) disclose side views of anchor insertion and control rods showing three alternative ways to control the insertion of the anchor allowing the anchor to be redirected in a non-linear method of insertion;

FIGS. 5(a)-5(c) are schematics disclosing a guide and flexible reamer or awl and method for creating the entry tunnel for the current invention;

FIGS. 6(a)-6(c) are schematics disclosing a control rod inside a guide and a method of insertion of the anchor at an angle different from the insertion angle of the control rod and guide;

FIGS. 7(a)-7(c) disclose side views of alternative embodiments of the anchor;

FIG. 8 depicts a side view of an alternate embodiment of the anchor with multiple sutures;

FIGS. 9(a)-9(c) depict side views of an alternate embodiments of the anchor with multiple suture holes and multiple sutures;

FIG. 10 discloses placement of the anchor in the intercondylar notch of the knee;

FIGS. 11(a) & 11(b) depict the insertion guide with a handle for insertion at varying angles;

FIGS. 12(a)-12(c) depict side views of alternate embodiments of the anchor with ridge angles and various attachment rods;

FIGS. 13(a)-13(c) disclose non-linear anchors inside guides or insertion devices that enable the anchor to engage the insertion tunnel in the bone or cartilage;

FIGS. 14(a) & 14(b) illustrate a cannulated embodiment with and without a guide wire; and

FIGS. 15(a)-15(d) are schematics illustrating how the cannulated embodiment is inserted by advancing the wire in the direction of the tunnel in the bone or cartilage.

FIGS. 16(a) & (b) respectively illustrate a side plan view and proximal plan view of a non-linear anchor for confined spaces in accordance with the present invention.

FIG. 17(a) illustrates a top view of the assembled anchor shown in FIG. 17(a).

FIGS. 17(b) & 17(c) respectively illustrate a side plan view and a proximal plan view of the mobile rotating component of the anchor disclosed in FIGS. 16(a) & 16(b).

FIG. 17(d) illustrates a side view of an alternate embodiment of the mobile rotating component of the anchor disclosed in FIGS. 16(a) & 16(b) with sharp threads.

FIGS. 18(a)-18(c) illustrate a top, side and rear views of the distal anchor member of the anchor disclosed in FIGS. 16(a) & 16(b).

FIGS. 19(a) & (b) respectively illustrate a side plan view and proximal plan view of an alternate embodiment of non-linear anchor in accordance with the present invention.

FIG. 20(a) illustrates a top view of the assembled anchor embodiment shown in FIG. 19(a).

FIGS. 20(b) & 20(c) respectively illustrate a side plan view and a proximal plan view of the mobile rotating component of the anchor disclosed in FIGS. 19(a) & 19(b).

FIG. 20(d) illustrates an alternate embodiment of the mobile rotating component of the anchor disclosed in FIGS. 19(a) & 19(b) with sharp threads.

FIGS. 21(a) & 21(b) illustrate an alternative embodiment of a non-linear anchor wherein the anchor folds or bends as it is inserted to the desired curved or non-linear shape. The figures show an embodiment with a toggle control rod, three sections, two hinges in both the unfolded, prior to insertion, and folded, after insertion, configurations.

FIGS. 22(a)-22(d) illustrate an alternative embodiment of a non-linear anchor system wherein the anchor is cannulated. These figures further illustrate the bend as it is inserted to the desired curved or non-linear shape. The figures show at least on loop or hook at one end of the anchor for suture attachment to the anchor. These are positioned to allow for free passage of a cannulated wire during insertion.

FIGS. 23(a)-23(i) illustrate various views of an anchor in accordance with an alternate embodiment of the present invention.

FIGS. 24(a) & 24(b), FIGS. 25(a), 25(b) & 25(b), FIGS. 26(a) & 26(b), and FIGS. 27(a), & 27(b) disclose various embodiments of control rod in accordance with the present invention.

FIG. 28 is a cross sectional view showing the anchor of FIGS. 23(a)-(i) being deployed in conjunction with the use of a control rod and an insertion device, wherein multiple transverse cross sectional views are provide a various positions along the length thereof.

FIG. 29 is a cross sectional view showing deployment of the anchor in accordance with the embodiment disclosed with reference to FIGS. 23(a)-23(i) using the control rod of FIGS. 27(a) & 27(b).

FIGS. 30, 31, 32 & 33 are cross sectional views showing deployment of the anchor in accordance with embodiment disclosed with reference to FIGS. 23(a)-23(i) using the control rod of FIGS. 27(a) & 27(b).

### DETAILED DESCRIPTION OF THE INVENTION

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

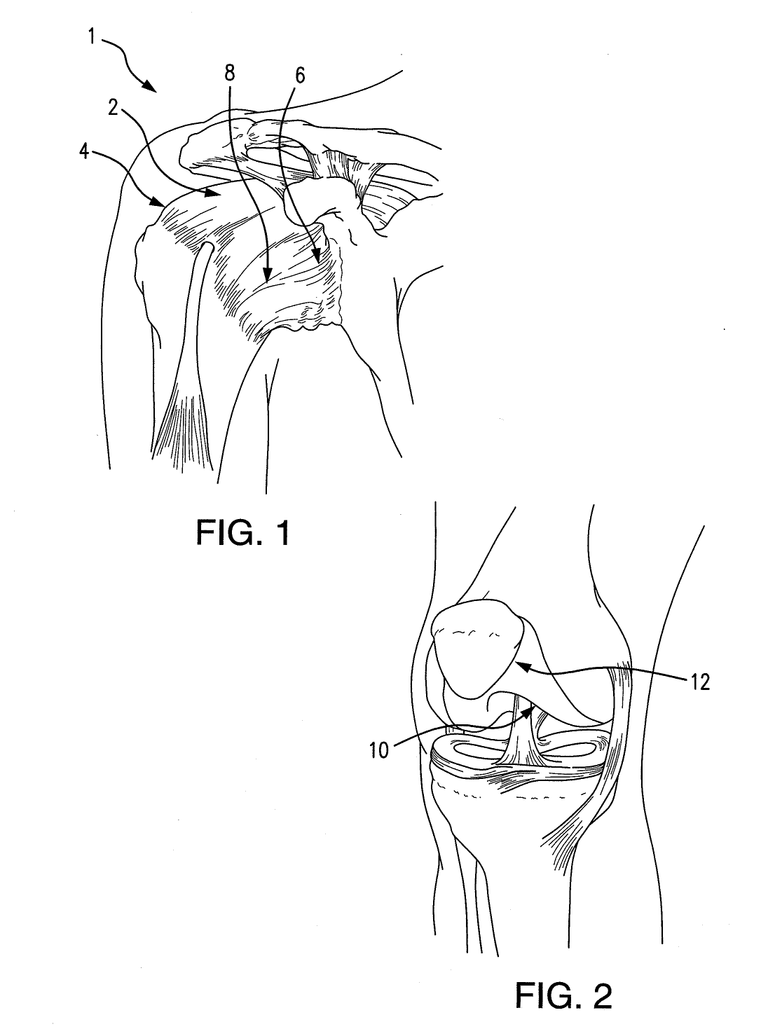

It has been determined by the present inventors that as surgical techniques have evolved and the ability to access confined spaces has improved, the absolute requirement of linear or co-linear application of these devices has become a distinct disadvantage in certain circumstances. This disadvantage is amplified in situations where the anatomy of the bone dictates the angle of the approach to the injured structure and the dictated angle is not ideal for the required application of the anchor or the repair. Efficient application is further complicated by the presence of nerves, arteries and other important soft tissue structures that confine the approach to the structure or structures requiring repair. For example, these complications arise in the subacromial space of the shoulder where the humeral head is partly covered by the acromion and the clavicle preventing a direct approach to the top of the humeral head (FIG. 1). This is also seen in the shoulder when approaching tears of the ligaments at the lower quadrants of the glenoid as in repairs for recurrent shoulder instability where a direct linear approach is limited by the axillary nerve and the brachial plexus and more medial approaches are limited by the coracoid process of the scapula, the musculocutaneous nerve and the conjoin tendon. This can also be seen in the confines of the intercondylar notch of the knee (FIG. 2), the acetabular labrum in the hip, as well as some endoscopic and arthroscopic approaches to other similar structures in a confined anatomic space.

More recently there has been a concern that the current linear systems force non-ideal placement of anchors and/or over tensioning of the soft tissues to bone or cartilage. This less than desirable positioning or tensioning is responsible for the high failure rates associated with certain repairs, for example, large rotator cuff tears with retraction of the torn tissue. In these cases, more medial placement of the anchors would reduce the tension on the repair, as is often done by prior non-endoscopic means.

Referring to the various figures and the embodiments disclosed therein, the present invention provides a suture anchor, a delivery assembly for inserting the anchor, and a method for attaching sutures to body tissue, such as bone or cartilage to repair injuries to tendons, ligaments, cartilage and other connecting structures in the body. The anchor is shaped and dimensioned for application in divergent or differing angles than the angle of the anatomically required approach. As a result, the anchor aids in improving the surgeon's ability to better approximate the open procedure endoscopically.

The present invention provides a suture anchor, or a fixation system, (along with a method of application) providing the ability of the anchor to hold one or more sutures. The anchor is preferably self-locking, allowing for knot tying or knotless applications for the use of biologic or non-biologic materials.

The anchor is applied in a non-linear, non-co-linear or a divergent angle to the insertion angle required to approach the anatomic structure as dictated by the confines of the local anatomy. The anchor is applicable to, but is not limited to, rotator cuff repairs, ligament repairs in the shoulder, repair of humeral avulsions of the geno-humeral ligaments, labrum or cartilage tears in the hip, and anterior cruciate ligament or knee ligament repair or reconstruction. It is appreciated the anchor is fabricated or produced from a number of biocompatible materials including, but not limited to, metals, plastic, polymers, bio-absorbable, biologic, or materials that are mechanically compatible with bone. For example, it is contemplated materials such as PEEK® polyether ether ketone thermoplastic would be well suited for use in accordance with the present invention. Furthermore, and as will be appreciated based upon the following disclosure, the present invention allows for newer procedures to be developed that have not been attempted in the past by virtue of the inability to access anatomic locations in a linear approach.

With the foregoing in mind, and as will be appreciated based upon the following disclosure, the present invention provides a method for attaching sutures to body tissue, such as bone or cartilage to repair injuries to tendons, ligaments, cartilage and other connecting structures in the body by application of a bone anchor in a non-linear, non-co-linear or a divergent angle to the insertion angle required to approach the anatomic structure as dictated by the confines of the local anatomy. The method is generally achieved by forming a tunnel or hole within an anatomical site and positioning a guide adjacent the tunnel or hole. The guide includes an angled outlet end having an opening transverse to the longitudinal axis of the guide. An anchor is passed through the guide to, and out of, the outlet end of the guide. The anchor is then positioned and secured within the tunnel or hole. Furthermore, this method takes advantage of the use of a curved anchor in a guide with a non-linear exit to achieve higher insertion angles than possible using the current state of the art and these angles are a requirement of anatomic repairs in the confined body spaces as encountered during advanced endoscopic surgery.

Referring to FIGS. 1 and 2, the anatomic restraints in the shoulder 1 that prevent subacromial approach to the medial humeral head 2, 4, approach to the gleno-humeral ligaments on the glenoid side 6 as in a Bankart repair or on the humeral side 8 as in an HAGL (Humeral Avulsion of the Gleno-humeral Ligaments), and the anatomic restraints to the intercondylar notch of the knee 10 and the under surface of the patella 12 are respectively shown.

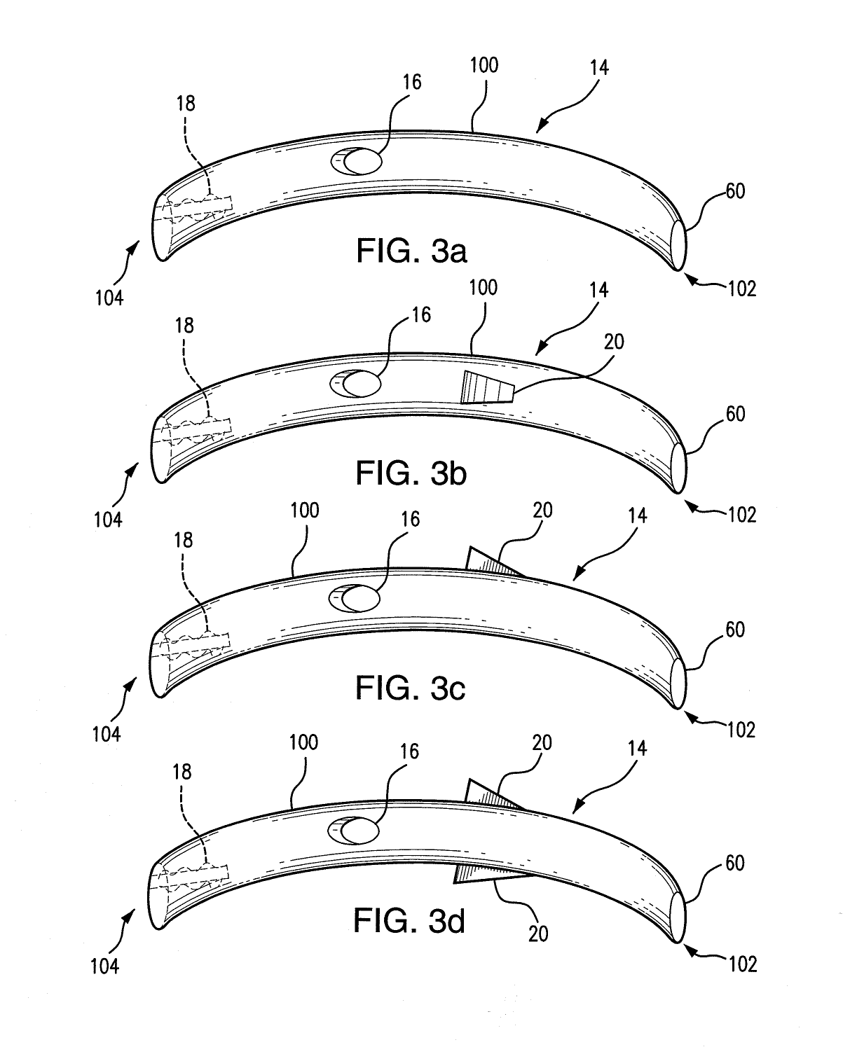

Referring to FIGS. 3(a), 3(b), 3(c) & 3(d), various embodiments of a curved suture anchor 14 are disclosed. The anchor 14 has an angle of curvature, that is, the arc defined by the curvature of the anchor 14, from between approximately 0 to 90 degrees, and more preferably between approximately 30 to 60 degrees. More particularly, the anchor may be provided with a 71 degree angle of curvature, a length of 15 mm and a width of 3 mm. It is also appreciated various applications concerning specific anatomic constraints might dictate greater angles of curvature or multiple angled bends in the anchor to allow redirection of the body of the anchor are disclosed. With this in mind, it is appreciated; a surgical kit could be provided offering a surgeon various anchor shape options, for example, 30, 45 and 60 degree angle of curvature anchors, for use during a medical procedure.

Each of the disclosed anchors 14 includes an elongated anchor body 100 with a first end 102 and a second end 104. At least one hole 16 is formed in the anchor body 100 at a centrally located position between the first end 102 and the second end 104. As will be appreciated based upon the following disclosure, the hole 16 is shaped and dimensioned for suture 48 placement. The anchor body 100 also includes an attachment site 18 for attachment of a control rod, or applicator rod, 24 at the second end 104 thereof. In accordance with the embodiment disclosed with reference to FIGS. 3(a)-(d), the attachment site 18 is a hole shaped and dimensioned for selective attachment to the control rod 24 for alignment to best take advantage of the curve or bend in the anchor to best facilitate insertion in a non-linear direction. In accordance with the one embodiment, the hole 18 is preferably threaded as shown in FIGS. 3(a)-(d), for mating with a similarly threaded distal end 108 of the control rod 24.

It is, however, appreciated, the hole may be cylindrical, conical or rectangular or hexagonal in shape to best hold the anchor until it is inserted and then release then anchor once seated. This may be facilitated by any of a number of mild adhesives if needed.

Referring to FIGS. 3(b), 3(c) & 3(d), the anchor body 100 of the anchor 14 may be provided with one or more “locking wings” 20. The locking wings 20 improve fixation once the anchor 14 is seated. FIG. 3(b) discloses a curved anchor 14 with a side locking wing 20. FIG. 3(c) discloses a curved anchor 14 with a top locking wing 20. FIG. 3(d) discloses a curved anchor 14 with a plurality of locking wings 20. The anchor body 100 is provided with a pointed tip 60 along the first end 102 thereof. The tip 60 is beveled to aid in application of the anchor 14.

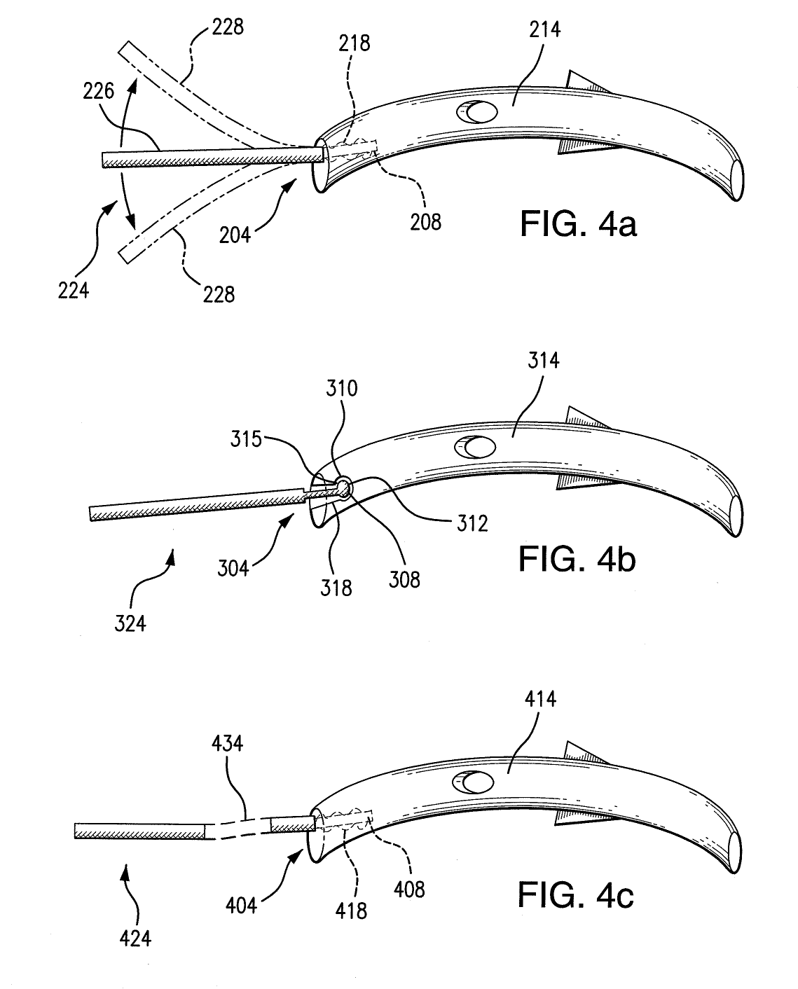

This versatility is further enhanced by the various embodiments of the flexible control rods 224, 324, 424 as disclosed with reference to FIGS. 4(a)-(c). These embodiments help to better control the movement and angulation of the anchor 14 as it is inserted or applied. Each of the disclosed attachment mechanisms allow for selective attachment of the distal end 208, 308, 408 of the control rod 224, 324, 424 to the attachment site 218, 318, 418 at the second end 204, 304, 404 of the anchor body 200, 300, 400.

Referring to FIG. 4(a), the distal end 208 of the control rod 224 is constructed of a flexible or malleable material allowing for a possible range of displacement of the control rod 224 as shown with reference to lines 226 and 228. The embodiment employs a threaded connection as discussed above with reference to FIGS. 3(a)-(c). One may use a spring, Nitinol wire or other spring like material, or a plastic with malleable or rubber like qualities.

In accordance with the embodiment disclosed with reference to FIG. 4(b), the anchor 314 and the control rod 324 are coupled via a ball joint 310. In particular, the distal end 308 of the control rod 324 is provided with a ball 312 shaped and dimensioned for selective coupling with a recess 315 formed in the second end 304 of the anchor 314. The recess 315 in the second end 304 of the anchor 314 is resilient biased to move between a closed configuration holding the ball 312 of the control rod 324 therein and an open configuration allowing for insertion and removal of the ball 312 from within the recess 315 at the second end 304 of the anchor 314. In addition to allowing for selective attachment, the ball joint 310 allows for relative movement between the control rod 324 and the anchor 314 allowing relative pivoting to the desired angle of approach for the anchor 314. This angle best serves the insertion process by being set at the complement of the angle of curvature of the anchor 314 and the desired insertion angle. For example, if there was a desire to insert the anchor 314 at 80 degrees to the angle of the entry port or cannula, and the angle of curvature of the anchor 314 is 55 degrees, the ball joint 310 opening would be designed to allow for 25 degrees of deflection on insertion yielding a turn of 55 degrees plus 25 degrees, for a total turn of 80 degrees. In a similar way, if a 45 degree insertion was required, an anchor 314 with an angle of curvature of 35 degree would require a 10 degree toggle or turn. An anchor 314 with a 64 degree angle of curvature with a 36 degree ball joint 310 would yield 100 degree insertion or application angle. In this manor, we can demonstrate any number embodiments with angles of insertion or application from 0 degrees to angles in excess of 90 degrees.

Referring to FIG. 4(c), the control rod 424 is provided with a flexible section or hinge 434 that allows for the desired angle of approach for the anchor 414. The embodiment employs a threaded connection as discussed above with reference to FIGS. 3(a)-(c). As in the ball joint embodiment shown with reference to FIG. 4(b), a wide range of hinge movement or angulations can be used to create a full range of insertion angles as demonstrated in the prior examples. However, it is appreciate the utilization of a hinge as disclosed above provides a control rod permitting bending only in one desired plane of motion as determined by the axis about which the hinge rotates. In addition, the hinge structure disclosed above is structured to limit the extent of rotational movement so as to limit the total bend angle to allow for optimal insertion of the anchor.

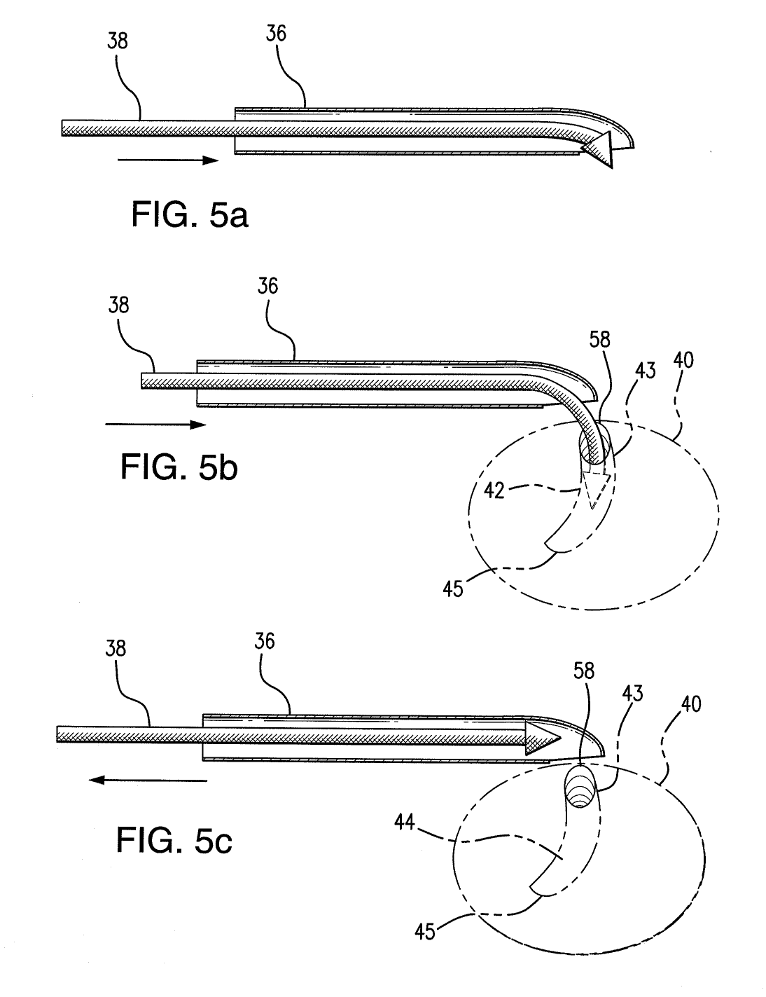

Prior to the application of an anchor 14 in accordance with the present invention, it will often be necessary to drill, ream or open a hole with an awl within the anatomical structure to which the anchor 14 is to be secured. FIGS. 5(a), 5(b) & 5(c) show a cylindrical reamer (or awl) guide 36, reamer (or awl) 38 and method for their use in accordance with the present invention. Referring to FIG. 5(a) the reamer 38 is positioned in the guide 36 and is set for an angled approach to the awl tunnel 44. In FIG. 5(b) the reamer 38 is advanced and in FIG. 5(c) the tunnel 44 is complete and the reamer 38 is removed. These figures show a methodology for drilling a tunnel or hole in the bone or cartilage at an angle different from the angle of the guide 36. It is this method that allows for placement of the curved anchor 14 as seen in the other figures.

As the FIGS. 5(b) & 5(c) show, the tunnel 44 is drill with a curved configuration having a radius of curvature similar to, but slightly less than the radius of curvature of the suture anchor 14. The tunnel 44 is also drilled such that the bottom 45 of the tunnel 44 is actually extending in the direction from which the guide 36 is accessing the bone or cartiledge 40; that is, and considering a plane extending normal to the bone surface at the far edge 43 of the entry to the tunnel 58 by the awl 38 and substantially perpendicular to the longitudinal axis of the guide 36, both the guide 36 (or at least a substantially portion thereof) during drilling and the bottom 45 of the tunnel 44 are on the same side of the plane.

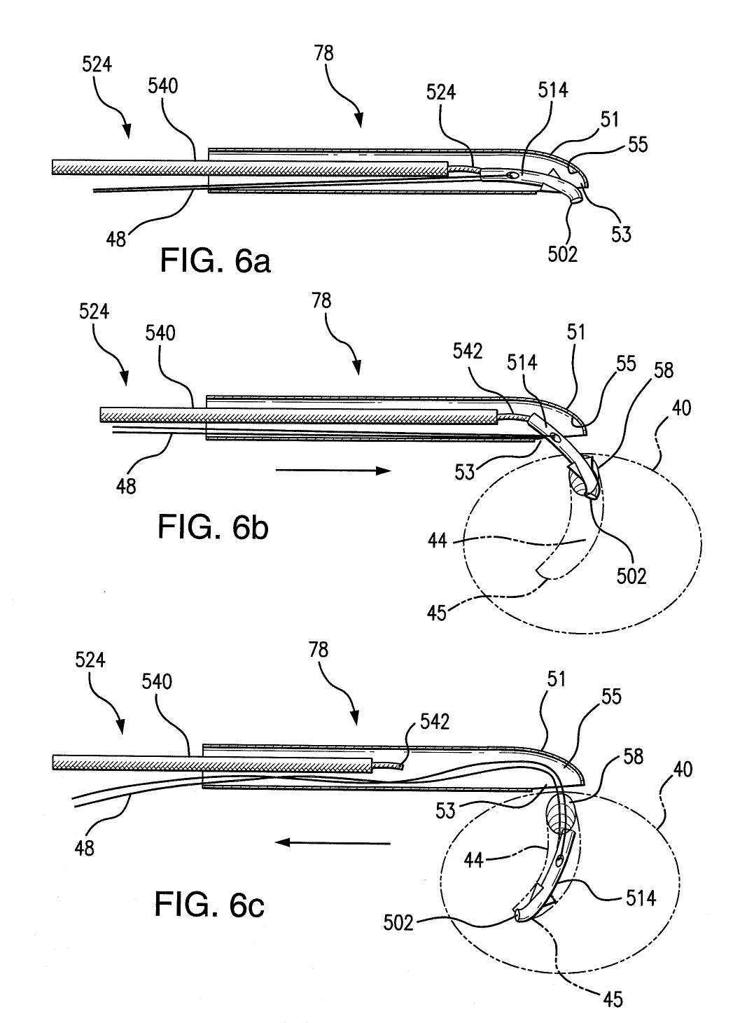

FIGS. 6(a), 6(b) & 6(c) show a procedure for placing the anchor 514 within the tunnel 44 at the desired anatomical site. While the anchor 514 and control rod 524 disclosed in conjunction with this embodiment are an alternate embodiment of those previously disclosed, it is appreciated the other embodiments disclosed herein may be employed in various combinations. The anchor 514 is coupled to the control rod 524, which is composed of a proximal rigid core 540 and distal flexible segment 542, adjacent the distal flexible segment 542. The anchor 514 is then is inserted into the pre-drilled tunnel 44 in the bone or cartilage 40. FIG. 6(a) shows the anchor 514, control rod 524 and a suture 48 contained in a cylindrical guide 78 with an angled distal end, or outlet end, 51 having an opening 53 transverse to the longitudinal axis of the guide 78, that is, the opening lies in a plane that is oblique or parallel in orientation relative to the longitudinal axis of the guide. The inner surface 55 of the guide 78 at the outlet end 51 is provided with a partial spherical shape. The spherical shape functions to direct the anchor 514 to the opening (or outlet) 53 at the outlet end 51 of the guide 78. In this embodiment, the assembly is complete with preloaded sutures 48. The assembly can be located over the entry to the tunnel 58 as seen in FIG. 6(b) and the control rod 524 is advanced demonstrating the method of anchor engagement in the entry hole 58 that permits the invention to move in a non-linear motion. The anchor 514 follows the curvature of the tunnel 44 upon entry and ultimately flips within the tunnel 44 such that the first end 502 of the anchor 514 actually extending in the direction from which the guide 78 is accessing the bone or cartiledge 40; that is, and considering a plane extending normal to the bone surface at the far edge 43 of the entry to the tunnel 58 and substantially perpendicular to the longitudinal axis of the guide 78, both the guide 78 (or at least a substantially portion thereof) and the first end 502 of the anchor 514 are on the same side of the plane when the anchor 514 is fully seated within the tunnel 44. In FIG. 6(c) the anchor 514 is fully seated and locked in the tunnel 44 with the sutures 48 in place and the control rod 524 is being removed. The “flipped” orientation of the anchor relative to the bone surface at the entry to the tunnel 58 assists in creating a secure seating arrangement. The sutures 48 are now free to be used in the application or surgical repair required.

In accordance with a preferred embodiment, the tunnel diameter is preferably 20% to 35% bigger than the anchor diameter. With this in mind, it is contemplated anchors with diameters of the 2.0 mm, 2.5 mm, 6.0 mm and 8.0 mm will be provided for use in conjunction with the present invention.

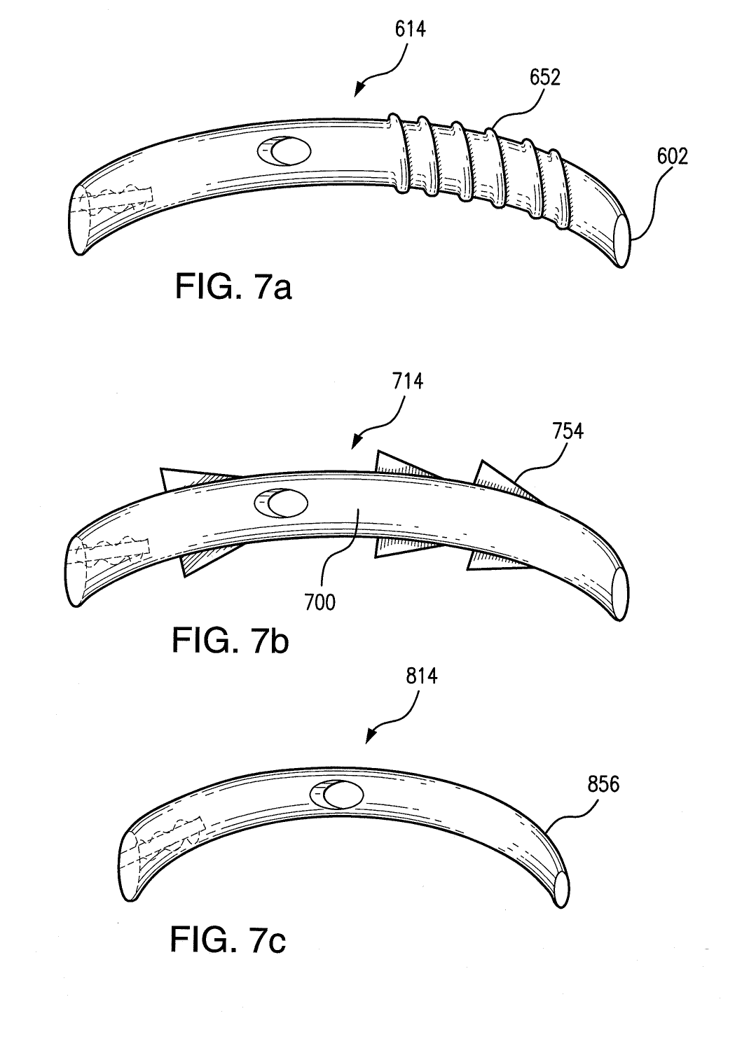

FIGS. 7(a), 7(b) & 7(c) disclose other embodiments of an anchor 614, 714, 814 in accordance with the present invention. FIG. 7(a) shows a curved anchor 614 with ridges or ribs 652 adjacent the first end 602 thereof. The ridges or ribs 652 increase the friction fit of the anchor 614.

FIG. 7(b) shows a curved anchor 714 with a plurality of wings 754 longitudinally spaced along the length of the anchor body 700. FIG. 7(c) shows an anchor 814 with a more acute radius of curvature as an alternate embodiment of the invention in the form of a non-linear anchor. In this way, and as described with reference to FIGS. 4(a)-(c), a full range of angled embodiments are possible.

Together FIGS. 5(a), 5(b) & 5(c) and FIGS. 6(a), 6(b) & 6(c) demonstrate a method of approach, chilling and application of alternate embodiments of a non-linear suture anchor as shown in FIGS. 3(a), 3(b) & 3(c) and FIGS. 7(a), 7(b) & 7(c) in a divergent hole or tunnel.

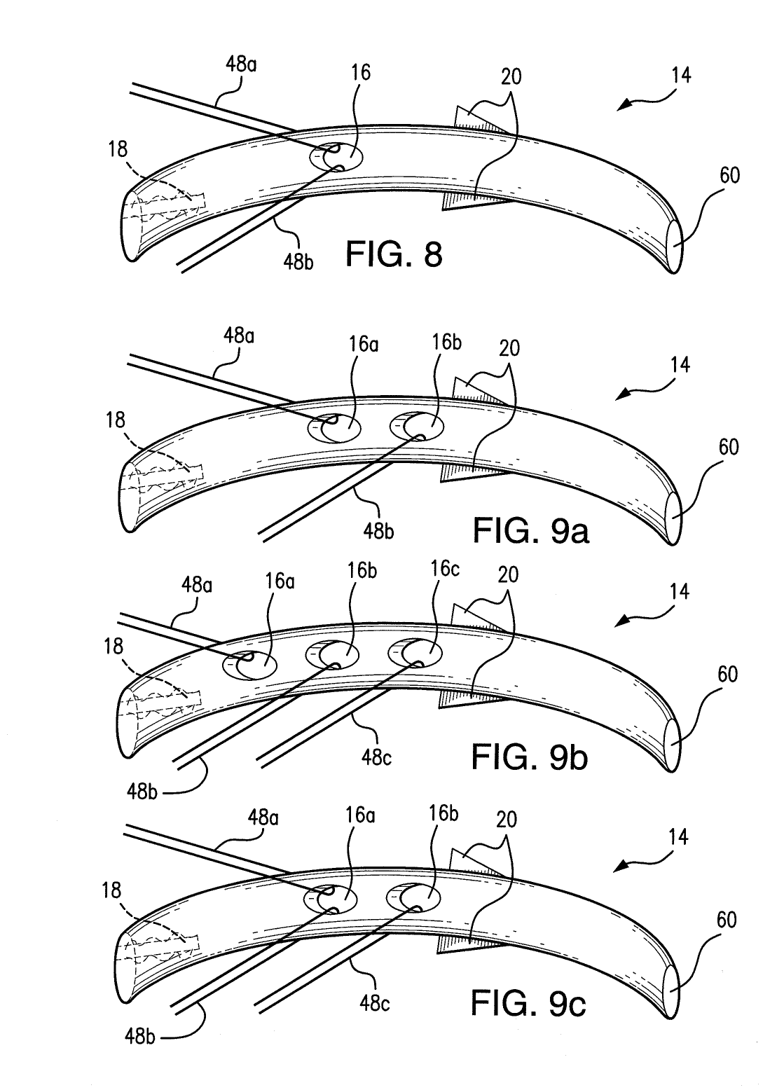

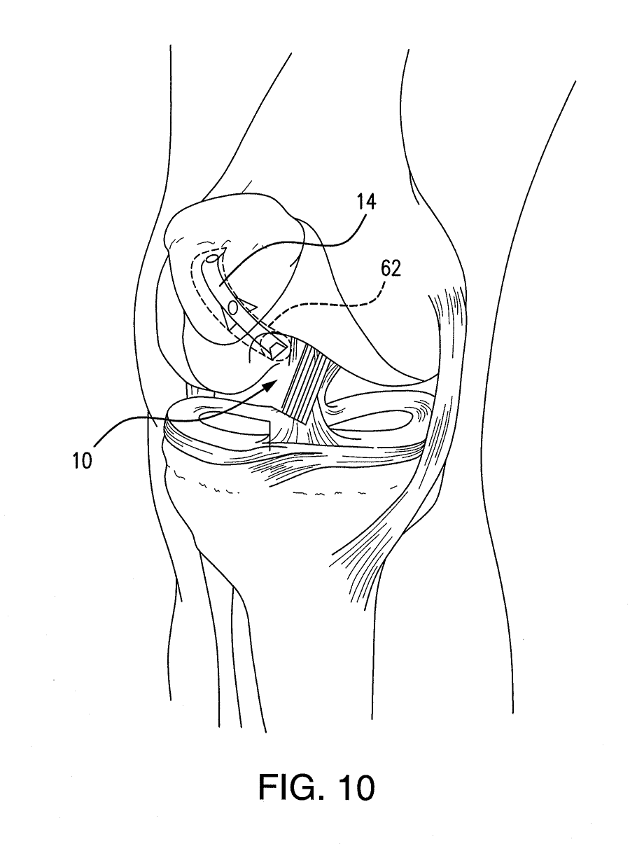

FIGS. 8, 9(a), 9(b) and 9(c) show alternate embodiments of the non-linear or curved anchor 14 with multiple sutures 48 passing through a single hole 16 (see FIG. 8) in the anchor 14 and multiple sutures 48 passing through multiple holes 16 in the anchor (see FIGS. 9(a)-9(c)). This demonstrates the ability of the non-linear or curved anchor 14 to accommodate multiple sutures 48 in a number of configurations that may be of benefit for a number of differing procedures. It should be appreciated, reference numerals for like elements shown in FIGS. 3(a)-(d) are employed in describing these embodiments.

More particularly, FIG. 9(a) shows first and second holes 16a, 16b with a first suture 48a passing through the first hole 16a and a second suture 48b passing through the second hole 48b. FIG. 9(b) discloses first, second and third holes 16a, 16b, 16c and respective first, second and third sutures 48a, 48b, 48c passing therethrough. Finally, FIG. 9(c) shows first and second holes 16a, 16b with first and second sutures 48a, 48b passing through the first hole 16a and a third suture 48c passing through the second hole 16b. These various configurations demonstrate a selection of embodiments with a varying number of holes and sutures contained in each hole. They are representative and the number of sutures and number of holes will be selected based upon the particular procedure.

FIG. 10 shows the insertion of the anchor 14 in a femoral tunnel 62 as one may use in an anterior or posterior cruciate ligament reconstruction. As will be appreciated, this may be achieved using any of the anchors or delivery mechanisms described herein. The non-linear or curved anchor 14 would not be restricted by the femoral condyle or the other structures in a trans-tibial or low medial approach and with the advantage of anatomic placement even with a more conventional medial portal placement for the insertion of the femoral ACL curved anchor.

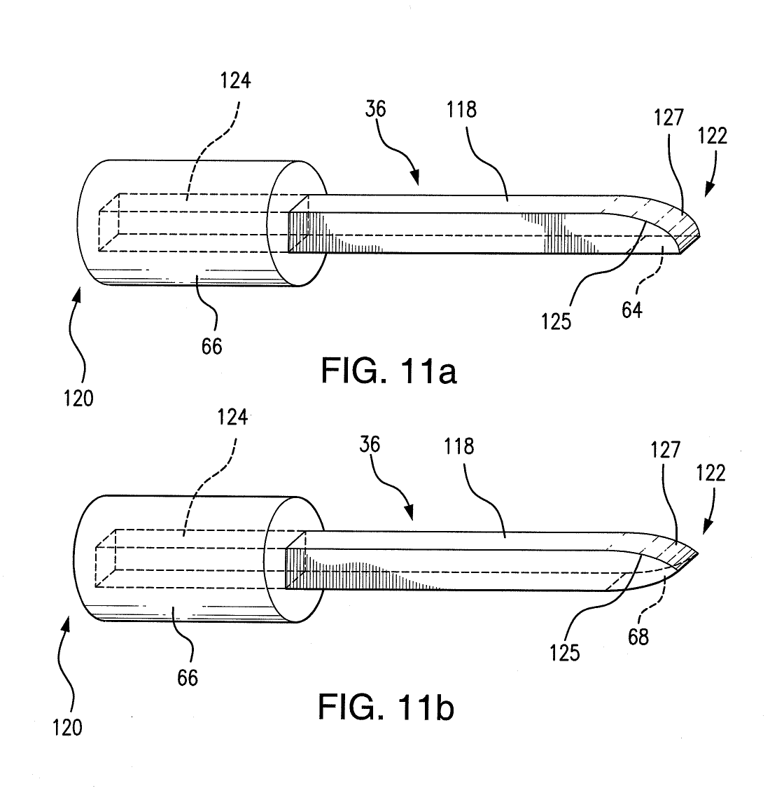

FIG. 11(a) shows a guide 36 for the insertion of the awl or anchor at approximately ninety degrees to the entry angle of the device through opening 64. The guide 36 is an elongated hollow member 118 having a first end 120 and a second end 122. A conduit 124 extends between the first end 120 and the second end 122 allowing for the passage of an awl or anchor in accordance with the present invention. The conduit 124 exhibits a substantially rectangular cross section when viewed in a plan perpendicular to the longitudinal axis thereof. The conduit 124 along the first end 120 and central portion of the guide 36 is substantially straight and parallel to the longitudinal axis of the guide 36. At the second end 122 of the guide 36, that is, at the outlet end of the guide 36, the conduit 124 turns such that the conduit 124, in particular, the inner surface 125 of the conduit 124 along the distal end 127 thereof, makes a substantially 90 degree turn allowing for deployment of the awl or anchor from the outlet or opening 64 at the second end 122 of the guide 36 in a direction substantially transverse, in particular, perpendicular, to the longitudinal axis of the guide 36. FIG. 11(b) discloses a guide 36 for insertion of the awl or anchor at an angle less than ninety degrees to the entry angle through opening 68. In particular, the outlet end of the conduit has an axis which is oblique to the longitudinal axis of the guide. This would vary between 30 and 60 degrees to be similar to the preferred angle of the anchor itself. For example an anchor with an angle of curvature of 45 degrees would have a guide with a 45 degree outlet end if it was on a straight control rod. Equally if an anchor having an angle of curvature of 45 degrees was on a 15 degree toggle or hinge than the preferred angle for the outlet end of the guide would be 45 degrees plus 15 degrees or 60 degrees. The guide for the awl may be a heavier construction to set the angle of approach for the flexible reamer and the guide for the anchor and control rod may be of lighter material. It is envisioned that the anchor and rod may be assembled or preloaded in a guide with or without a handle 66 and with or without sutures for ease of insertion.

The anchor may be made of varying sizes, lengths and curves to accommodate specific uses or procedures. For example, the anchor may be small for a glenoid labrum procedure, larger for a rotator cuff repair, and even larger for larger ligament repairs like an anterior cruciate ligament in the knee.

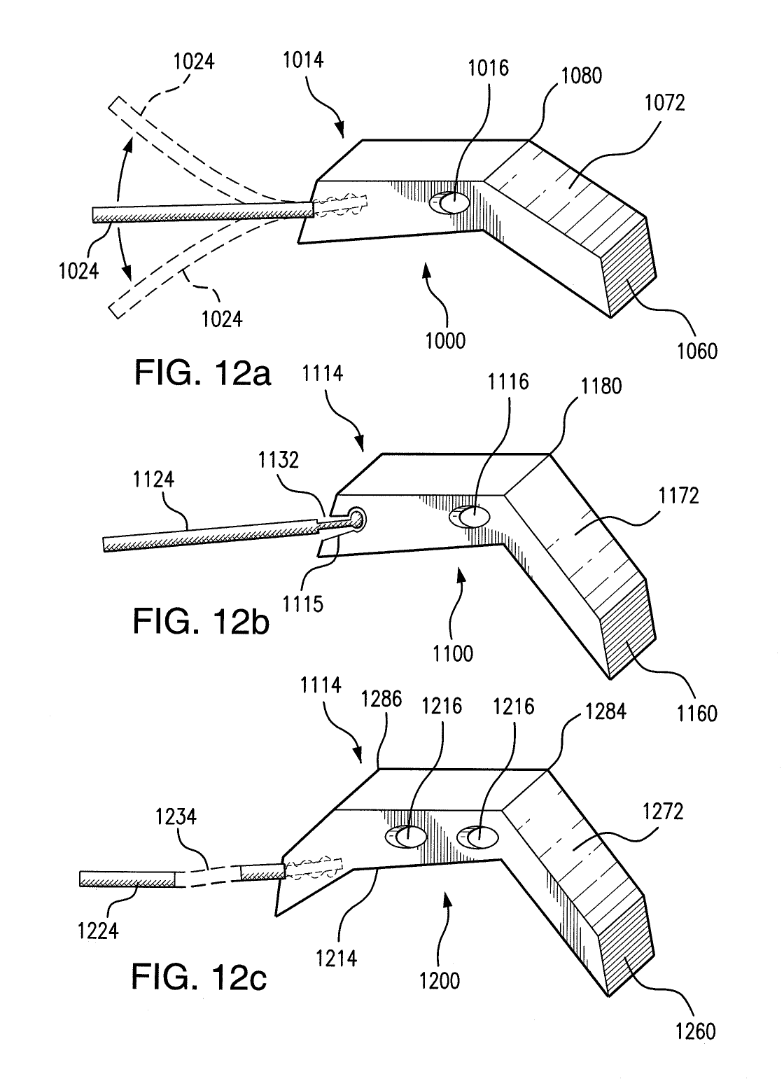

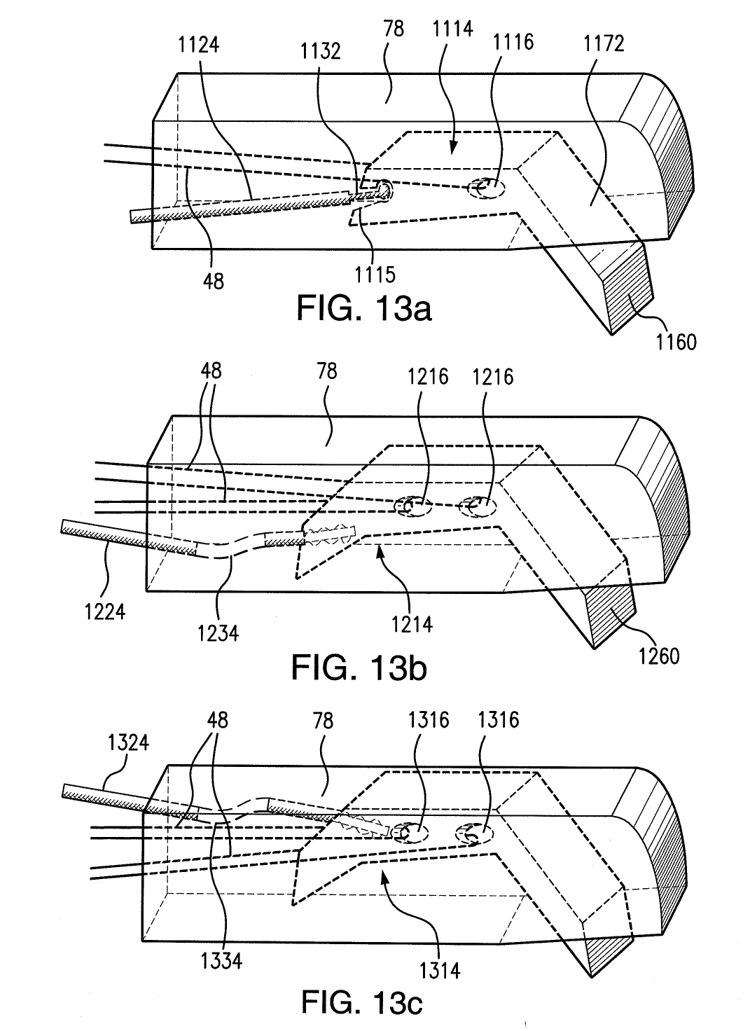

Referring to FIGS. 12(a), 12(b) & 12(c), additional embodiments of the non-linear anchor 1014, 1114, 1214 having one or more fixed angles in the anchor body 1000, 1100, 1200 of the anchor 1014, 1114, 1214 to create a self-seating property with an insertion device end 1072, 1172, 1272 are disclosed. These non-linear anchors 1014, 1114, 1214 may be deployed with control rod 1024 similar to that disclosed with reference to the embodiment disclosed in FIG. 4(a), control rod 1124 similar to that disclosed with reference to the embodiment disclosed in FIG. 4(b), or control rod 1224 similar to that disclosed with reference to the embodiment disclosed in FIG. 4(b). The insertion device end 1072, 1172, 1272 allows the tip 1060, 1160, 1260 of the anchor 1014, 1114, 1214 to protrude out of the side of the guide 78 (see FIGS. 13(a)-(c). The tip is then used to locate the awl hole and aid in centering the anchor over the hole for insertion.

In FIGS. 12(a)-(c) three different non-linear anchors 1014, 1114, 1214 with rigid angles, instead of a curved body, are shown. As discussed above, the anchor and control rod coupling constructions are similar to those disclosed respectively with reference to FIGS. 4(a), 4(b) & 4(c) to control the insertion angle of the anchor allowing the anchor to be redirected in a non-linear method of insertion. FIG. 12(a) shows a flexible or malleable control rod 1024 and an anchor 1014 with a single angle 1080 with a hole 1016 for at least one suture. FIG. 12(b) shows a ball joint 1115 attachment site for a more rigid control rod 1124 with an angled entry in the mounting section 1132 of the anchor 1114 with a single angle 1180 (more acute than that disclosed in FIG. 12(a)) and a hole 1116 for at least one suture to allow for non-linear insertion of the anchor 1114. FIG. 12(c) shows a control rod 1224 with hinge or flexible section 1234 to allow for a multi-angled anchor 1214 with angles 1284, 1286 for non-linear insertion of the anchor 1214 with more than one hole 1216 for at least one suture in each hole 1216 and a more acute angle 1284 of the anchor 1214 with a second angle 1286 in the anchor 1214 making a more significant bend to enable more secure fixation and an even higher angle of approach. Any combination of the control rods and the anchors are possible pending the required application of the device.

In FIGS. 13(a)-(c) different non-linear anchors as shown with reference to FIGS. 12(a)-(c) are disclosed inside guides or insertion devices 78 that allow the tip of the anchor 88 to engage the insertion hole, as depicted in FIG. 6(a)-(c), in the bone or cartilage 40. FIG. 13(a) discloses the anchor 1114 and control rod 1124 of FIG. 12(b), as well as one suture 48 passing through the hole 1116. FIG. 13(b) shows the anchor 1214 and control rod 1224 of FIG. 12(c) having a flexible hinge 1234 at the distal end of the control rod 1224 and more than one hole 1216 having more than one suture 48 passing therethrough. The device is used to locate the awl hole and advance the anchor into position in a manner similar to that depicted in FIG. 6. In other embodiments, as seen in FIG. 13(c), the control rod 1324 is provide with a distal end 1334 having the ability to bend in multiple directions providing the anchor 1314 with alternative positions 90 for the guide or control rod to aid in having the anchor seat in the hole and lock in place.

The deployment assembly for the anchor can be loaded or preloaded or pre-assembled with the tip of the anchor protruding varying amounts to use the tip of the anchor as a hole finder for starting the anchor in the hole and seating it in place. The indirect way of locating the hole is advantageous. In this way the deployment assembly and anchor assembly will act as an aid in finding the hole for proper placement. With this anchor assembly and deployment assembly, the anchor can be then seated with less difficultly in more challenging locations.

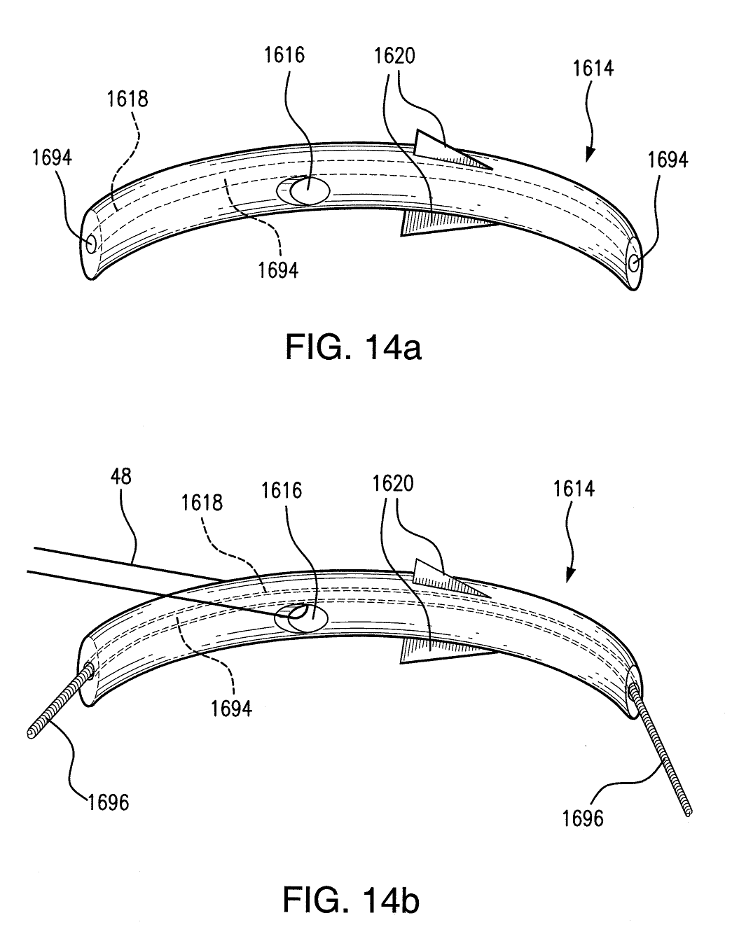

It is further envisioned that the non-linear anchor of the present invention may be produced with a hole down its long axis, with side loops or internally cannulated so it is applied in a non-linear or indirect fashion using a flexible or malleable guide wire. For example, FIG. 14(a) shows yet another embodiment of an anchor including a cannulation 1694 down the center of the anchor body 1600 of the anchor 1614. As with the prior embodiment, this anchor also includes at least one hole 1616, locking wings 1620 and an attachment site 1618. As shown in FIG. 14(b), the cannulation 1694 provides a passageway for the use of a guide wire 1696 during deployment of the anchor 1614.

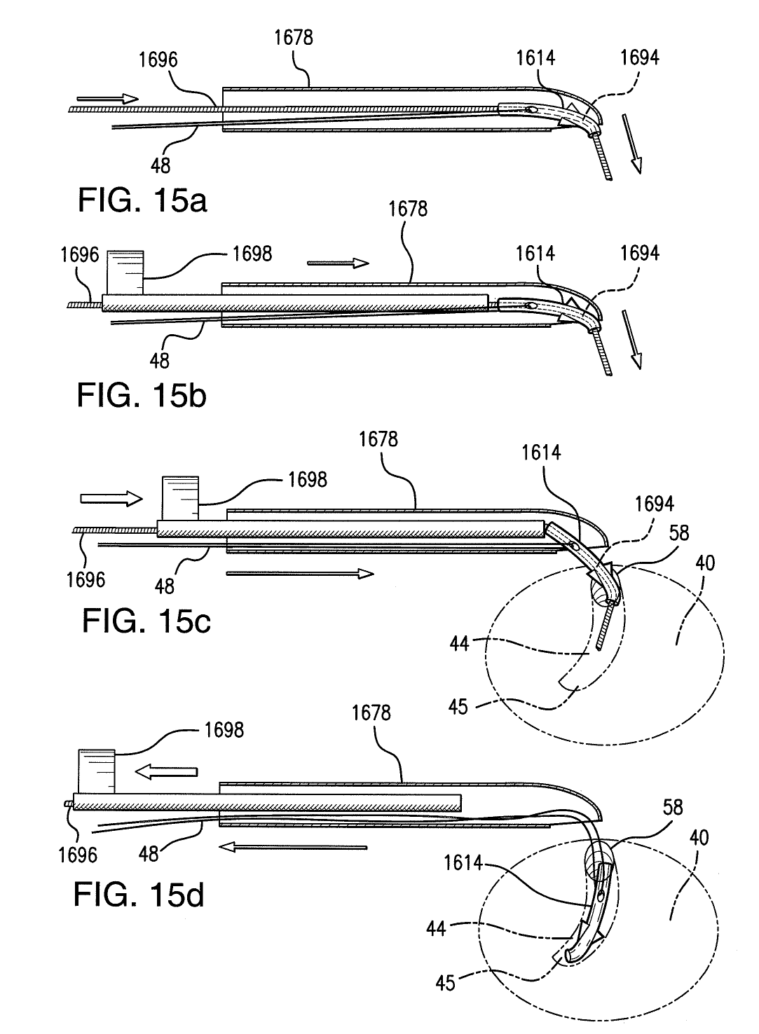

Referring to FIGS. 15(a)-(d) a possible method of insertion using a guide wire as part of the technique is disclosed. FIG. 15(a) shows the cannulated anchor 1614 with a guide wire 1696 passing through the cannula 1694 as the anchor 1614 extends through a guide 1678 (similar to those guide discussed above with regard to FIGS. 6(a)-(c)). FIG. 15(b) shows the guide wire 1696 advanced in the direction of the tunnel 44 in the bone or cartilage 40. As with the embodiment disclosed with reference to FIGS. 5(a)-5(c) and FIGS. 6(a)-6(c), the hole is curved to enhance anchor seating and placement. A flexible of malleable pusher 1698 is secured to the guide wire 1696 for moving it into position. In FIG. 15(c), the pusher 1698 is advanced so the anchor 1614 enters the tunnel 44 and in FIG. 15(d) the anchor 1614 is shown in place with removal of the insertion device 1678 and pusher 1698 with the sutures 48 in place.

It is appreciated the non-linear or curved anchors of the present invention are not limited to systems of press fit fixation in the bone, wing fixation or active expanding wings or barbs. Because of the different qualities of bone as determined by age, sex, bone quality and bone density, the present invention provides embodiments that can be adapted specifically for screw-in design capabilities and that can accommodate multiple thread patterns to best secure the anchor to the bone of the patient.

With this in mind, it is appreciated the non-linear anchor described herein may be adapted to a screw-in mechanism for bone fixation. In this way, the application of the anchor to the bone would not be limited to the type of bone, cortical or cancellous, for optimum fixation or pull out strength. The threads of the threaded sections as described below in greater detail can vary in pitch, shape, pattern and size. The two piece anchor disclosed below may be adapted for ease of manufacture and allow one anchor to accommodate many thread configurations. At the same time these anchors can be applied in a non-linear method in a constrained space, something not possible using the anchors described in the current state of the art.

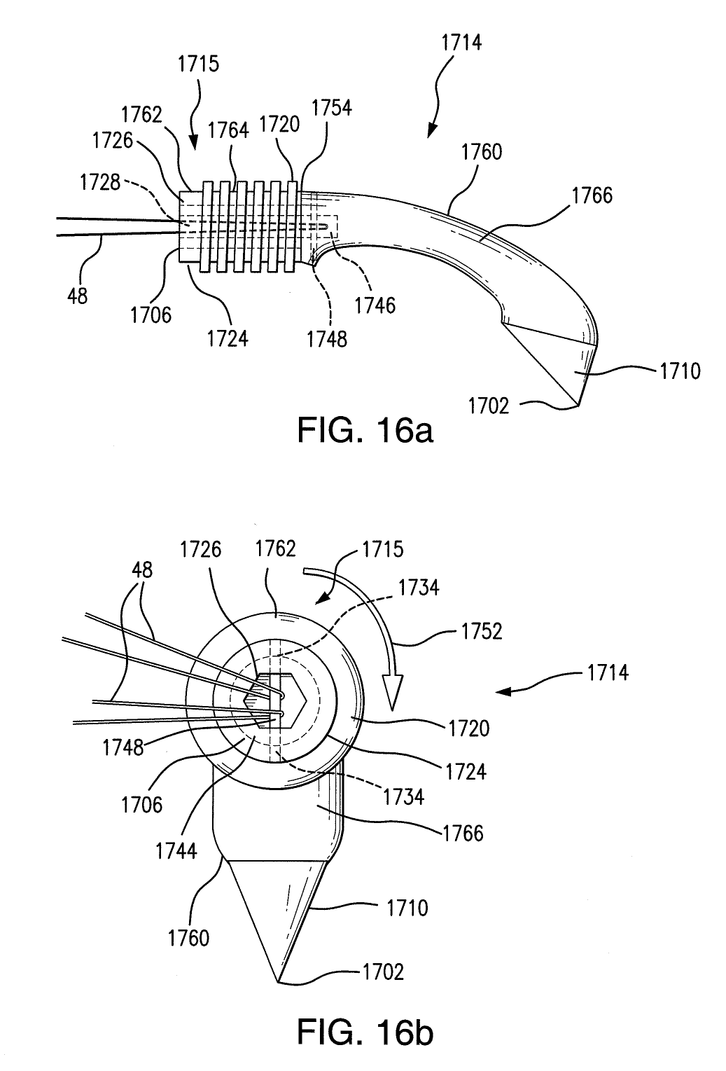

Referring now to the embodiment shown in FIGS. 16(a) & (b), 17(a)-(d) and 18(a)-(c), a non-linear, curved anchor 1714 with a screw-in locking mechanism 1715 is disclosed. The anchor 1714 is constructed in two-pieces, that is, a distal anchor member 1760 and a proximal anchor member 1762. As will be appreciated based upon the following disclosure, the distal anchor member 1760 and the proximal anchor member 1762 are secured for relative movement allowing for rotation of the proximal anchor member 1762 relative to the distal anchor member 1760 in a manner permitting a medical practitioner to take advantage of the threads 1720 formed along the exterior surface 1764 of the proximal anchor member 1762 by screwing the proximal anchor member 1762 into bone.

The anchor 1714 is self guided into place due to the shape of distal anchor member 1760. In particular, the distal anchor member 1760 is composed of a curved anchor body 1766 and a tip 1710 which function to guide the anchor 1714 into position.

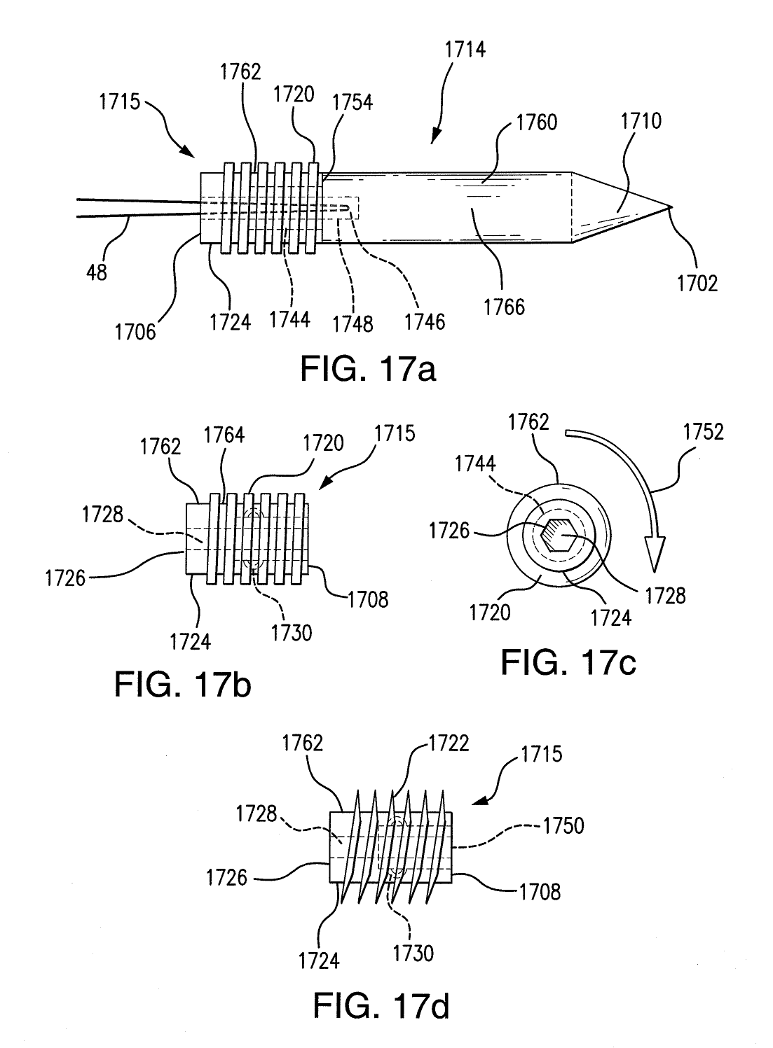

In accordance with a disclosed embodiment, the proximal anchor member 1762 is secured to the distal anchor member 1760 in a manner permitting the proximal anchor member 1762 to be turned clockwise 1752 to lock in place when threads engage bone thereby taking advantage of threading 1720 formed along the exterior surface 1764 of the proximal anchor member 1762. The threads can be in many forms, pitches and shaped such as square edged threads 1720 (see FIGS. 17(a)-(c)) and sharp edges 1722 (see FIG. 17(d)). The addition of the threads increases the fixation strength in certain applications while providing a bone capturing mechanism that avoids the use of active wings, ribbing, or barbs to secure the anchor to bone.

As discussed above, the anchor 1714 includes a proximal anchor member 1762 and a distal anchor member 1760. Consequently, the anchor 1714 includes a distal end 1702, a proximal end 1706, a curved anchor body 1766, a mobile rotating component 1724, wherein the distal anchor member 1760 includes the distal end 1702 of the anchor 1714 and the curved anchor body 1766 and the proximal anchor member 1762 includes the proximal end 1706 of the anchor 1714 and the mobile rotating component 1724.

The mobile rotating component 1724 includes an exterior surface 1764 with outwardly extending threads 1720 (or 1722) and a hole 1728 extending through its center. The hole 1728 at the proximal end 1726 of the mobile rotating component is, in accordance with a preferred embodiment, hexagonal shaped to accommodate a driver. In particular, a driver is shaped and dimensioned to securely fit within the hole 1728 at the proximal end 1726 of the mobile rotating component 1724 and rotate the mobile rotating component 1724 for engaging the threads 1720 with the adjacent bone. Within the mobile rotating component 1724, the hole 1728 widens as it extends to the distal end 1708 of the mobile rotating component 1724. The hole 1728 at the distal end 1708 contains an inner track (or rim) 1730 shaped and dimensioned to accommodate and match an outer ridge 1732 formed on an inner shaft 1744 extending from the proximal end 1754 of the of the distal anchor member 1760. These are constructed so the mobile rotating component 1724 can freely rotate on the proximal end 1726 of the distal anchor member 1760 around the inner shaft 1744 extending from the proximal end 1754 of the distal anchor member 1760.

The inner shaft 1744 includes a hole 1746 within its center. The hole 1746 includes at least one inner suture bar (or peg) 1748 attached to the distal anchor member 1760 within the hole 1746 of the inner shaft 1744. The suture bar 1748 provides a structure for attachment of one or more sutures 48 to the anchor 1714. The suture bar 1748 may have a smooth shape to allow sutures to slide in order to ease passing the sutures and tying a variety of knots. The suture bar 1748 and suture 48 are seen in FIG. 16(a) and one possible multiple suture arrangement can be best seen through the hexagonal opening of hole 1726 located at the proximal end 1706 in FIG. 16(b).

The inner shaft 1744 is constructed of a resilient material and includes a split 1734. The split 1734 allows ready assembly of the anchor 1714 by permitting one to slightly squeeze the inner shaft 1744 and slide the hole 1728 at the distal end 1708 of the mobile rotating component 1724 over the inner shaft 1744 of the proximal anchor member 1762. The mobile rotating component 1724 will “pop” over the ridge 1732 to lock the mobile rotating component 1724 onto the inner shaft 1744 on the distal anchor member 1760 by fitting the inner track 1730 about the ridge 1732. This creates a threaded assembly where the mobile rotating component 1724 is freely mobile and can spin on the inner shaft 1744 (that is, the distal anchor member 1760) while the proximal anchor member 1762 and the distal anchor member 1760 act as a complete anchor 1714. The anchor 1714 can then be inserted as a unit into the bone guided by the tip 1710 and the curve of the anchor body 1766. Once the tip 1710 and anchor body 1766 are engaged, the mobile rotating component 1724 could be turned in direction 1752 advancing the anchor 1714 and seating it into the bone. At least one suture 48 would be anchored to the bone via the suture bar 1748 and freely siding within the center hole or channel 1746 of the proximal anchor member 1714 and its inner shaft 1744.

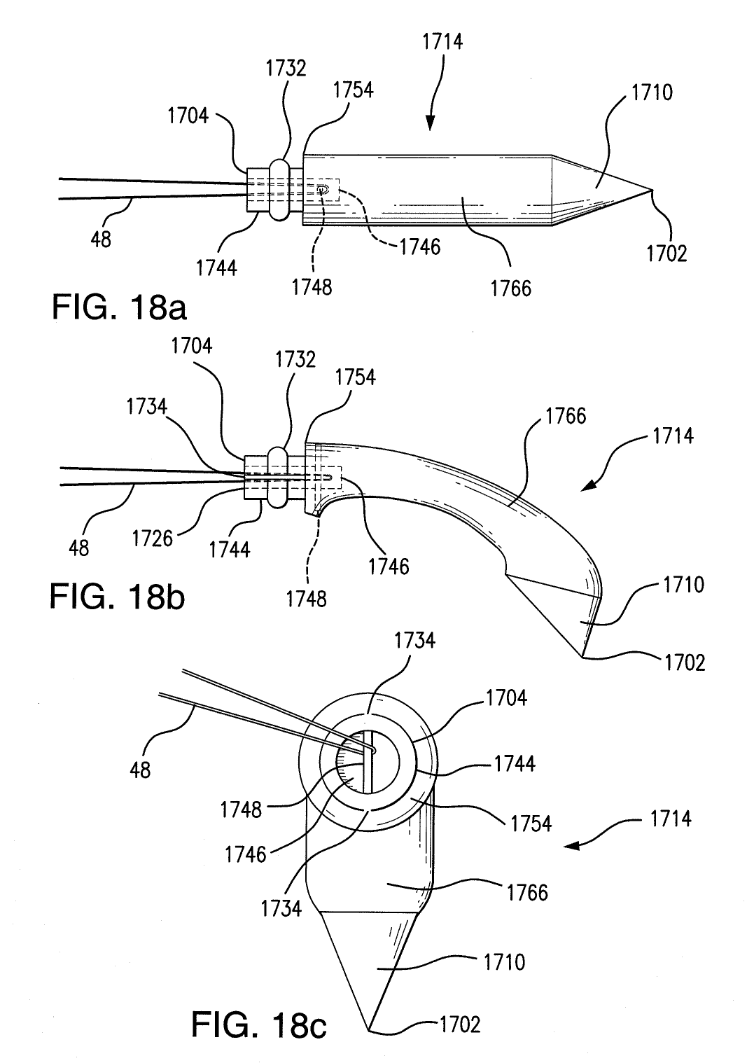

The hole 1728 of the mobile rotating component 1724 extends fully therethrough and the mobile rotating component 1724 includes an opening at each of the proximal end 1726 and the distal end 1708 thereof. The mobile rotating component 1724 contains an inner track or groove 1730 holding the proximal end 1706 and the distal end 1708 together. In this preferred embodiment, at the proximal end 1726 the hole has a hexagonal shape to accept a flexible cannulated screw driver into the hexagonal shaped hole 1728 at the proximal end 1726 of the mobile rotating component 1724. On the distal end 1708, the hole 1728 is large enough to accommodate the inner shaft 1744 of the distal anchor member and its ridge 1732 as discussed above. The outer ridge 1732 on the inner shaft 1744 mates (FIG. 18(a)) and can turn on the inner groove 1730 inside the mobile rotating component 1724 (FIG. 17(d)). The outer threads 1720 can be square or sharp 1722 or another shape best suited for the bone type. The thread pitch may be varied and the size of the thread or thread dept may also vary. In this embodiment the sutures are secured inside the body of the anchor 1714 around the suture bar 1748. The inner shaft 1744 has at least one split 1734 (FIG. 18(a)) to allow the edges to compress together. This split should be of sufficient size to allow the mobile rotating component 1724 to pop onto the inner shaft 1744 until the distal end 1708 of the proximal anchor member 1762 and the proximal end 1754 anchor body 1766 are in close proximity and remain associated for insertion. Once assembled the threaded section turns freely on the anchor.

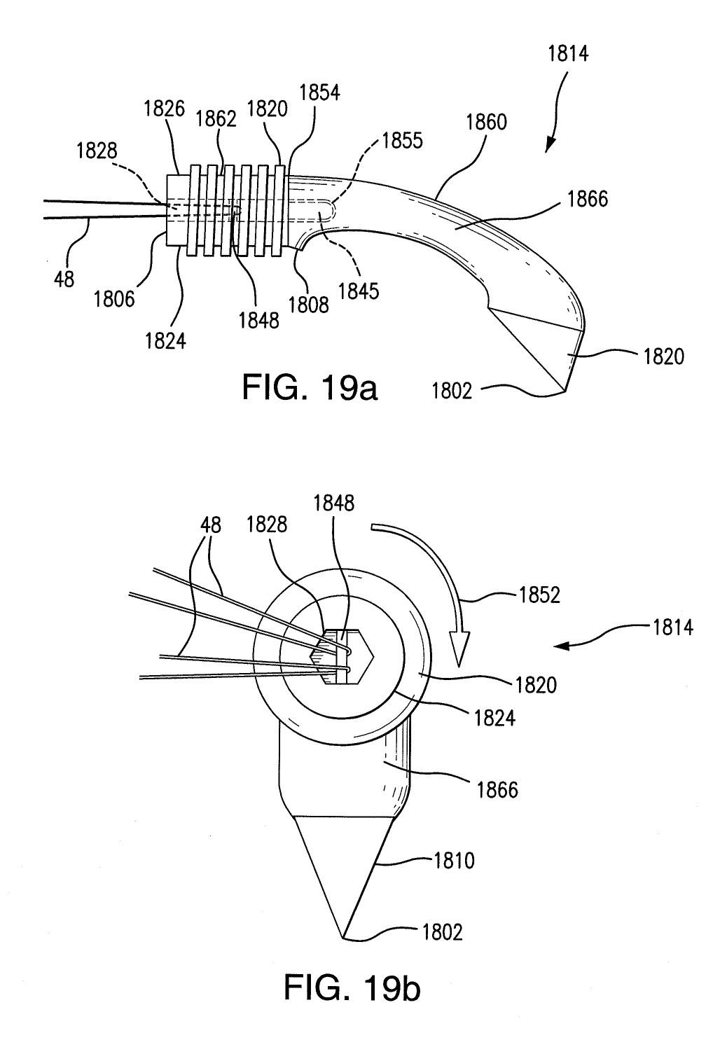

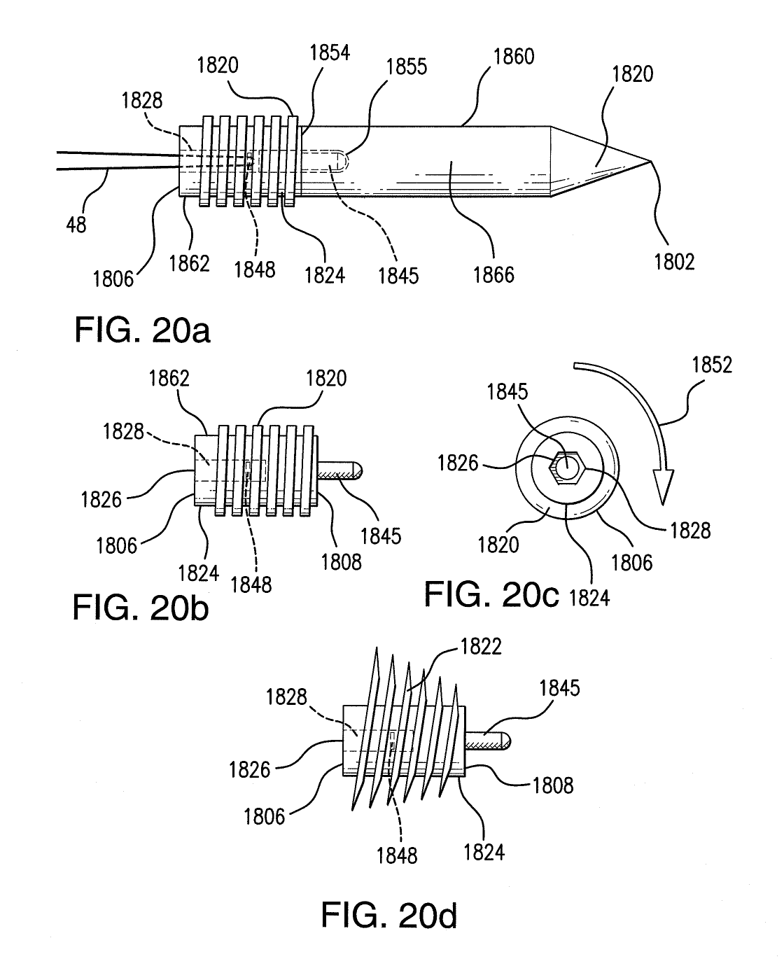

FIGS. 19 and 20 show an alternative embodiment of the anchor disclosed above with reference to FIGS. 16(a) & 16(b), 17(a)-(d), and 18(a)-(c), wherein the anchor 1814 includes a distal end 1802, a proximal end 1806, a curved anchor body 1866, and a mobile rotating component 1824, wherein the distal anchor member 1860 includes the distal end 1802 and the curved anchor body 1866 and the proximal anchor member 1862 includes the proximal end 1806 and the mobile rotating component 1824. In contrast to the embodiment disclosed above with reference to FIGS. 16(a) & (b), 17(a)-(d), and 18(a)-(c), the inner shaft 1744 is replaced with a hole 1855 in the proximal end 1854 of the distal anchor member 1860 and the mobile rotating component 1824 includes a post, peg or pole 1845 extending from the distal end 1808 thereof for engagement with the hole 1855 in a manner securing the proximal anchor member 1862 and the distal anchor member 1860 together in a manner permitting relative rotation.

The suture bar 1848 is positioned in the body of the mobile rotating component 1824 instead of the body of the distal anchor member 1760 as disclosed with reference to the embodiment of the FIGS. 16(a) & 16(b), 17(a)-(d), and 18(a)-(c) and the sutures pass inside the hole 1828 formed in the proximal end 1826 of the mobile rotating component 1824 through the proximal end 1826 thereof which is exhibits a hexagonal shape. The junction between the distal anchor member and the proximal anchor member could be affixed with a break-away adhesive, glue or partial attachment of the two parts that snap, give or break as the threaded section is turned in direction 1852 once the anchor is seated to the level of the threaded section.

The non-linear anchor of the present invention may also fold into shape allowing a linear insertion device to place the anchor in a non-linear location. The anchor then conforms to the desired angle as it folds into its final configuration. These flexible anchors can have at least one screw-in mechanism or threaded section for bone fixation. The self-folding anchor may be attached to a flexible, rigid, or toggle inserter to direct the non-linear insertion.

The non-linear or curved anchor system described herein may further be adapted to a flexible body as apposed a rigid body. The flexible anchors of the present invention can have at least one screw-in mechanism or threaded section for bone fixation. In this way, the application of the anchor to the bone would not be limited to the type of bone, cortical or cancellous, for optimum fixation or pull out strength. The threads of the threaded section described in this preferred embodiment can vary in pitch, shape, pattern and size. The flexible anchor may be cannulated to direct the non-linear insertion.

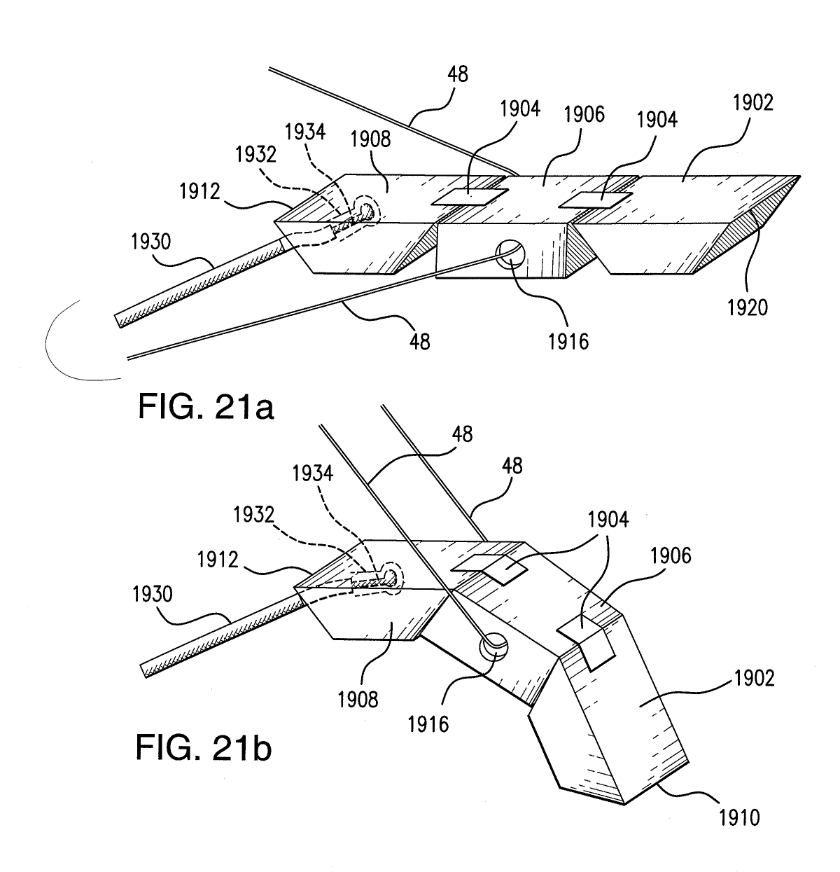

It is appreciated the invention may also be non-linear in a further configuration as shown in FIG. 21(b) and linear prior to insertion as illustrated in FIG. 21(a). In accordance with the embodiment disclosed in FIGS. 21(a) & 21(b), the anchor has a distal end 1910 and a proximal end 1912. The self folding anchor has at least two sections, and, in accordance with a preferred embodiment as disclosed in FIGS. 21(a) & 21(b) includes first anchor section 1902, second anchor section 1906, and third anchor section 1908. The first anchor section 1902, second anchor section 1906, and third anchor section 1908 are coupled with hinges 1904 permitting relative movement.

In accordance with a preferred embodiment, the center section, that is, the second anchor section 1906 has at least one hole 1916 formed therein. The hole 1916 is shaped and dimensioned for receipt of at least one suture 48. It is, however, appreciated there may be more than one hole 1916 in any of the first, second or third anchor sections 1902, 1906, 1908.

The proximal end 1912 of the anchor, that is, the proximal end of the third anchor section 1908, has a recess 1932 shaped and dimensioned for engagement with a ball member 1934 at the end of a control rod 1930 as discussed above with reference to the embodiment shown in FIG. 5(b). As such, the anchor and the control rod are coupled via a ball joint.

In addition, it is appreciated the ledge edge 1910, that is, the distal edge of the first anchor section 1902 may be curved, beveled or pointed to assist in the insertion of the anchor in a non-linear manor.

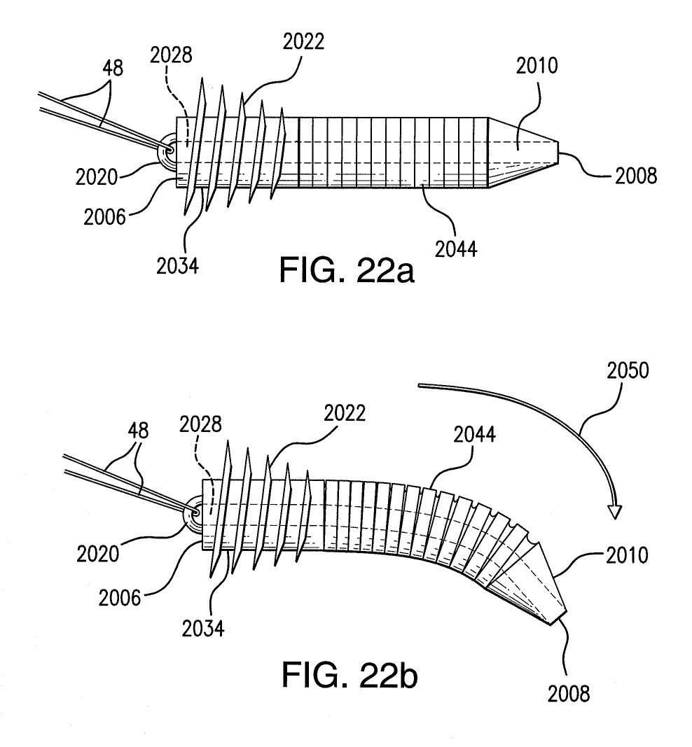

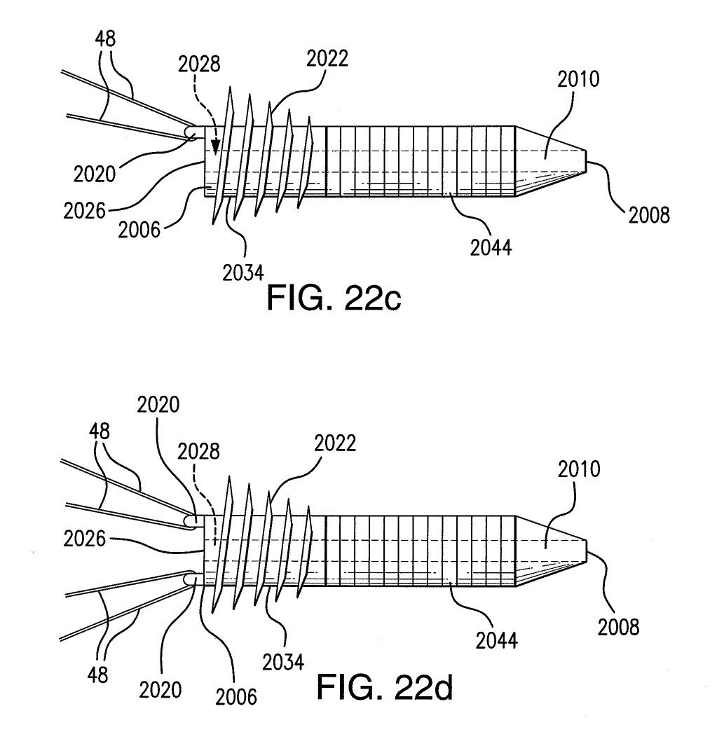

Referring now to the embodiment disclosed with reference to FIGS. 22(a)-(d), the anchor has a distal end 2008 and a proximal end 2006. The length between the proximal end 2006 and the distal end 2008 can be divided into sections, that is, proximal anchor section 2034, a central anchor section 2044, and a distal anchor section 2010. With this in mind, the central anchor section 2044 is made of a flexible material resulting in a flexible section capable of being bent into a desired shape as shown in FIG. 22(b).

In accordance with a preferred embodiment, the anchor is formed with the preferred embodiment shown has three sections, that is, the distal anchor section 2010, the central anchor section 2044, and the proximal anchor section 2034. The distal anchor section 2010 is tapered, the central anchor section 2044 bends or is flexible, and the proximal anchor section 2034 is threaded. There is a cannulation from the distal end 2008 to the proximal end 2006 for a flexible wire. The proximal end 2006 has at least one eyelet 2020 at the proximal end positioned to hold sutures but not interfere with the central cannulation 2028, these are best seen in FIGS. 22 (a) and 22(b) in a side view and in FIGS. 22(c) and 22(d) in a top view. In addition, the proximal anchor section 2034 is formed with external threads 2022 (which may be sharp as shown or be square edged threads as discussed above). This mechanism would increase the fixation strength in certain applications while providing a bone capturing mechanism that avoids the use of active wings, ribbing, or barbs to secure the anchor to bone. In this preferred embodiment, at proximal end 2006 there is a hexagonal hole 2026 to accept a flexible cannulated screw driver into the hexagonal hole 926.

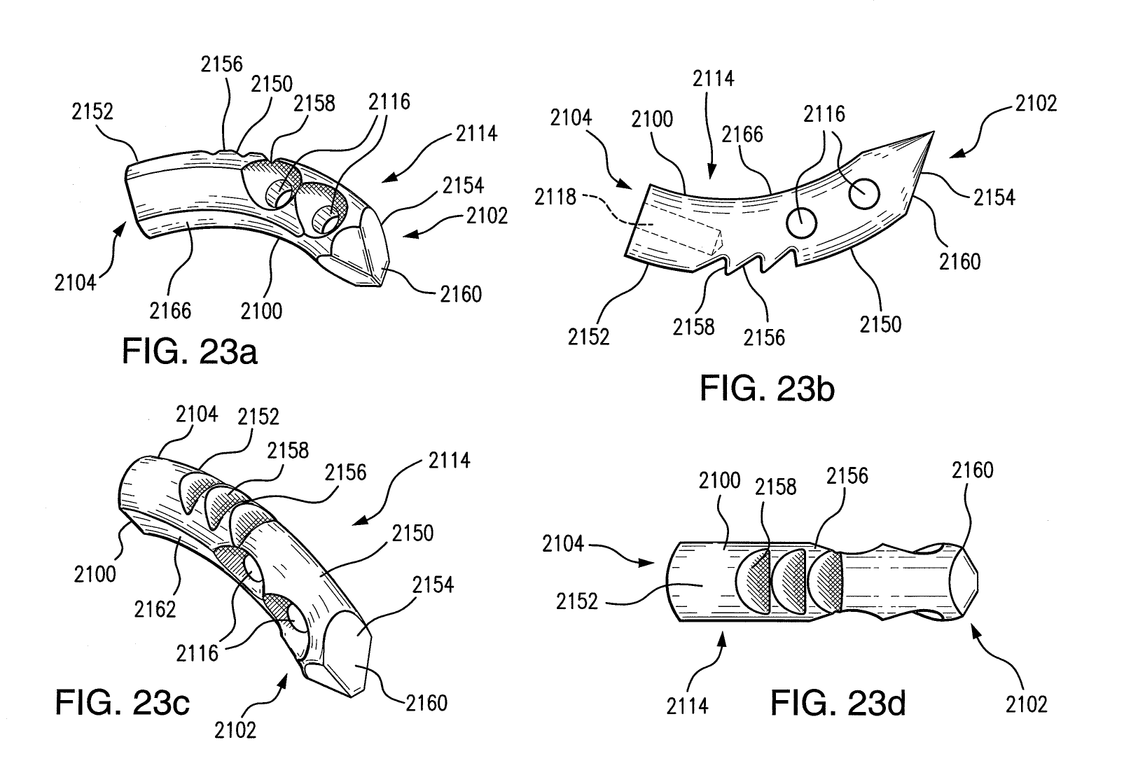

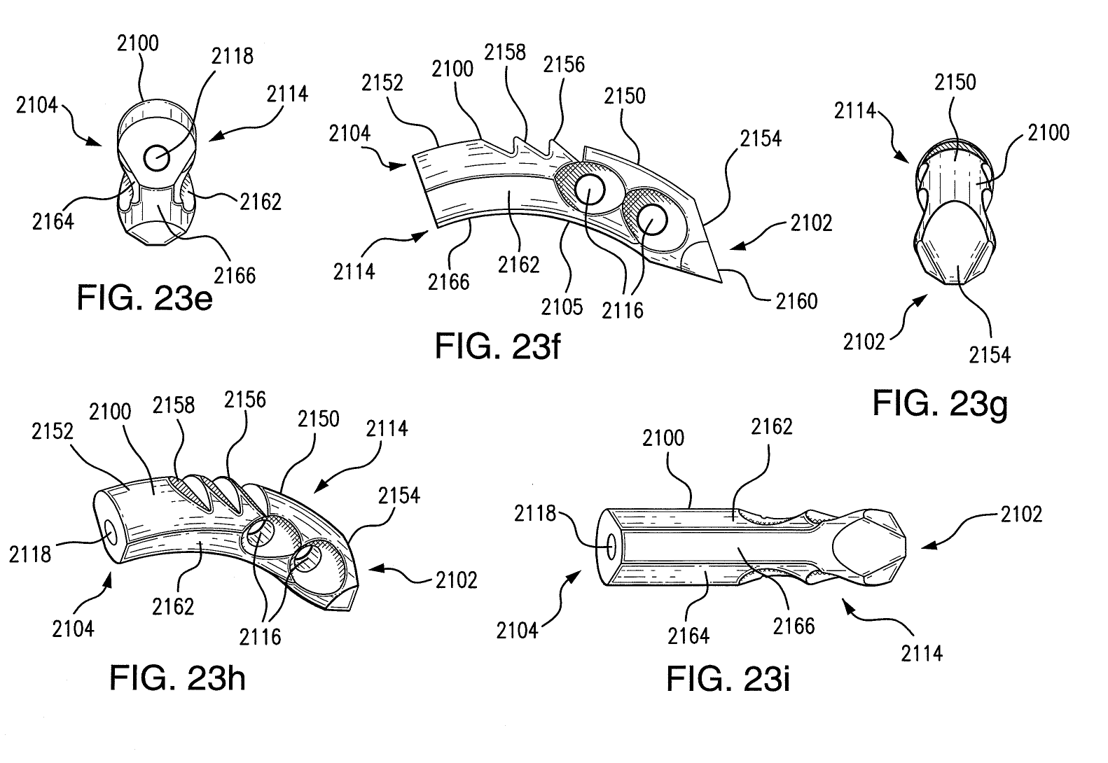

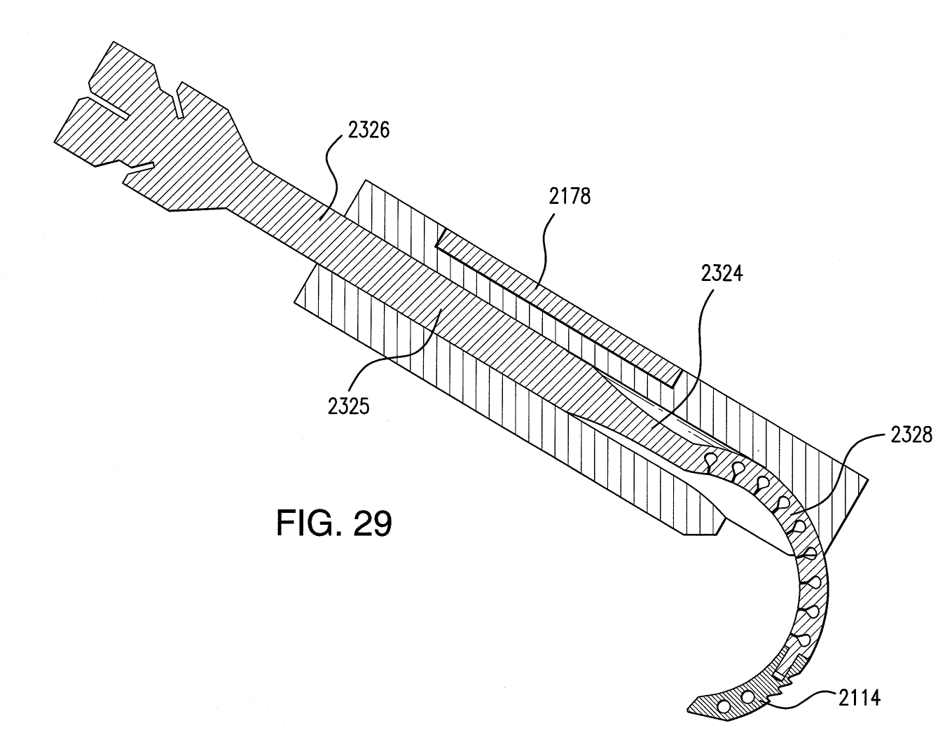

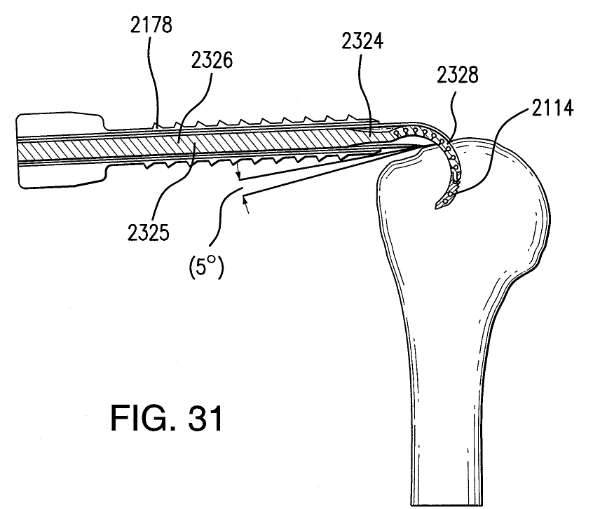

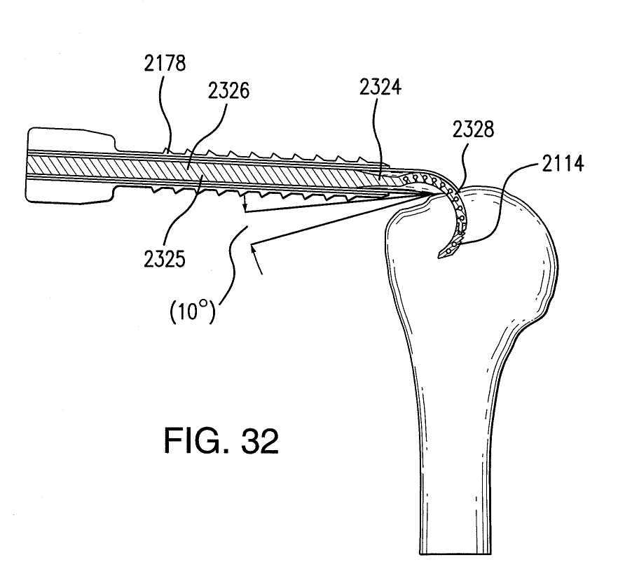

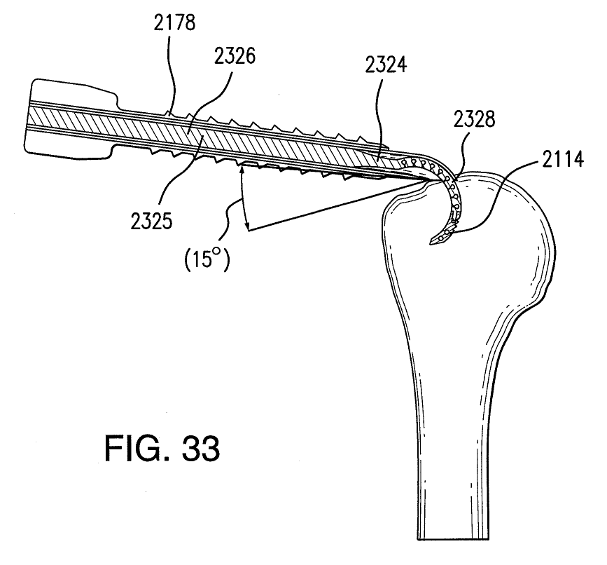

In accordance with yet another embodiment of the present invention, and with reference to FIGS. 23(a)-(i), 24(a) & 24(b), FIGS. 25(a), 25(b) & 25(b), FIGS. 26(a) & 26(b), FIGS. 27(a) & 27(b), and FIGS. 29 to 33, alternate embodiments for the construction of the anchor 2114, control rod 2124, 2224 and insertion device 2278 is disclosed. The disclosed embodiment provides triangular, or pie-shaped, configuration enhancing the functionality thereof.

With specific reference to the anchor 2114, the anchor 2114 includes an elongated anchor body 2100 with a first end 2102 and a second end 2104. At least one suture hole 2116 is formed in the anchor body 2100 at a centrally located position between the first end 2102 and the second end 2104. As with the prior embodiments, the hole 2116 is shaped and dimensioned for suture placement. The anchor body 2100 also includes an attachment site 2118 for attachment of the control rod 2124 at the second end 2104 thereof. As with the embodiments discussed above, the attachment mechanism may take a variety of forms previously discussed with regard to the prior embodiments.

As with the embodiment shown with reference to FIGS. 3(a)-(d), the suture anchor 2114 exhibits a curved configuration as it extends from the first end 2102 to the second end 2104. The anchor 2114 has an angle of curvature, that is, the arc defined by the curvature of the anchor 2114, from between approximately 0 to 90 degrees, and more preferably between approximately 30 to 60 degrees. Adjacent the first end 2102 of the anchor 2114 the tip 2160 is provided with a sharper angled surface. More particularly, the anchor body 2100 is provided with a convex top surface 2150 and concave bottom surface 2152 as the respective arcs extend from the first end 2102 to the second end 2104 of the anchor body 2100.

With reference to the convex top surface 2150, it includes a proximal segment 2152 adjacent the second end 2104 of the anchor body 2100, a distal segment 2154 adjacent the first end 2102 of the anchor body 2100, and a central segment 2156 between the proximal segment 2152 and the distal segment 2154. Sharp unidirectional grooves 2158 are laterally formed along the convex top surface 2150 of the anchor body 2100, and extend along the central segment 2156 of the convex top surface 2150.

The convex top surface 2150 offers a relatively consistent profile as it extends along the proximal segment 2152 and the central segment 2156. However, the distal segment 2154 is formed so as to offer a stepper angle of decent as it extends toward the tip 2160 at the first end 2102 of the anchor body 2100. As such, the angle between the central segment 2156 and the distal segment 2154 is between approximately 120 degrees and 170 degrees. This steeper angle sets the guide position on the bone.

As briefly discussed above, the anchor body 2100 includes a substantially triangular shape when viewed along a cross section taken perpendicular to a longitudinal axis of the anchor body 2100. As such, the anchor body 2100 further includes a convex top surface 2150 as discussed above. The convex top surface 2150 includes a convex arcuate shape as it extends from the first end 2102 to the second end 2104 of the anchor body 2100. The convex top surface 2150 also includes a convex arcuate shape as it extends from the first and second lateral sides 2162, 2164 of the anchor body 2100.

The first and second lateral sides 2162, 2164 of the anchor body 2100 extend from the convex top surface 2150 at an acute angle such that they meet at the concave bottom surface 2166 of the anchor body 2100. The concave bottom surface 2166 includes a concave arcuate shape as it extends from the first end 2102 to the second end 2104 of the anchor body 2100. The concave bottom surface 2166 also includes a convex arcuate shape (that is, rounded) as it extends from the first and second lateral sides 2162, 2164 of the anchor body 2100.

The suture holes 2116 are positioned so as to extend through the anchor body 2100 from the first lateral side 2162 thereof to the second lateral side 2164 thereof at a position between the top surface 2150 and the bottom surface 2166. The provision of the suture holes 2116 on the thinner or narrower part of the anchor 2114 helps to lock the anchor 2114 in place and help with the turn and gliding features of the lead sutures. It is contemplated that one suture hole or more than two suture holes could be provided in accordance with the invention.

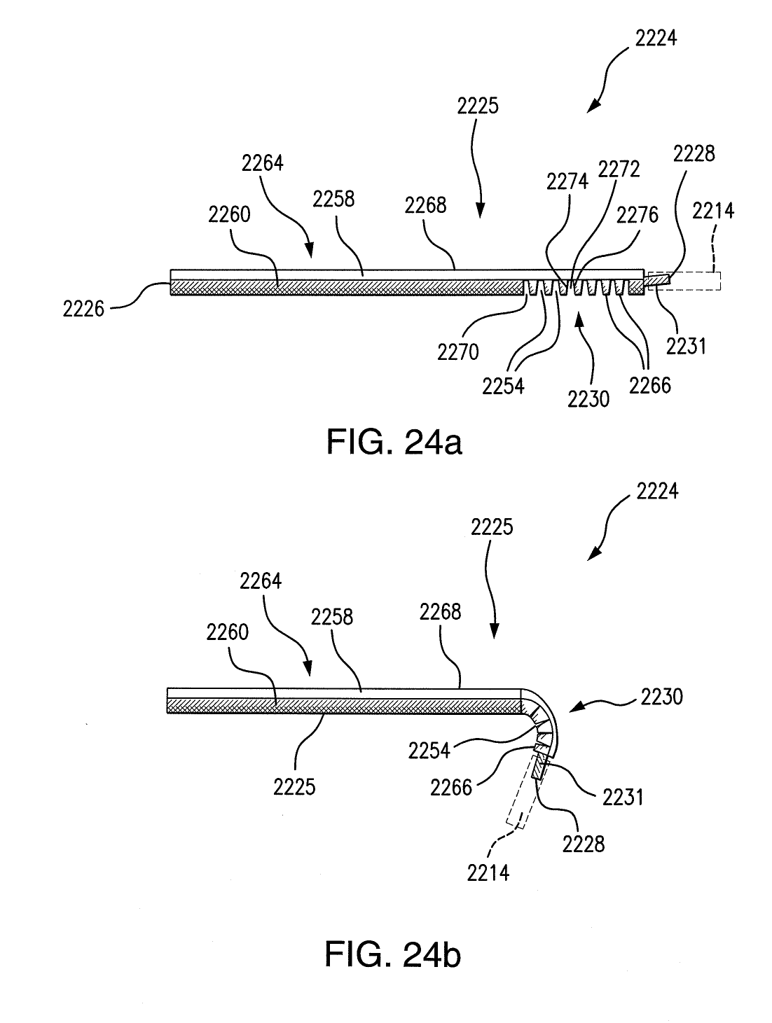

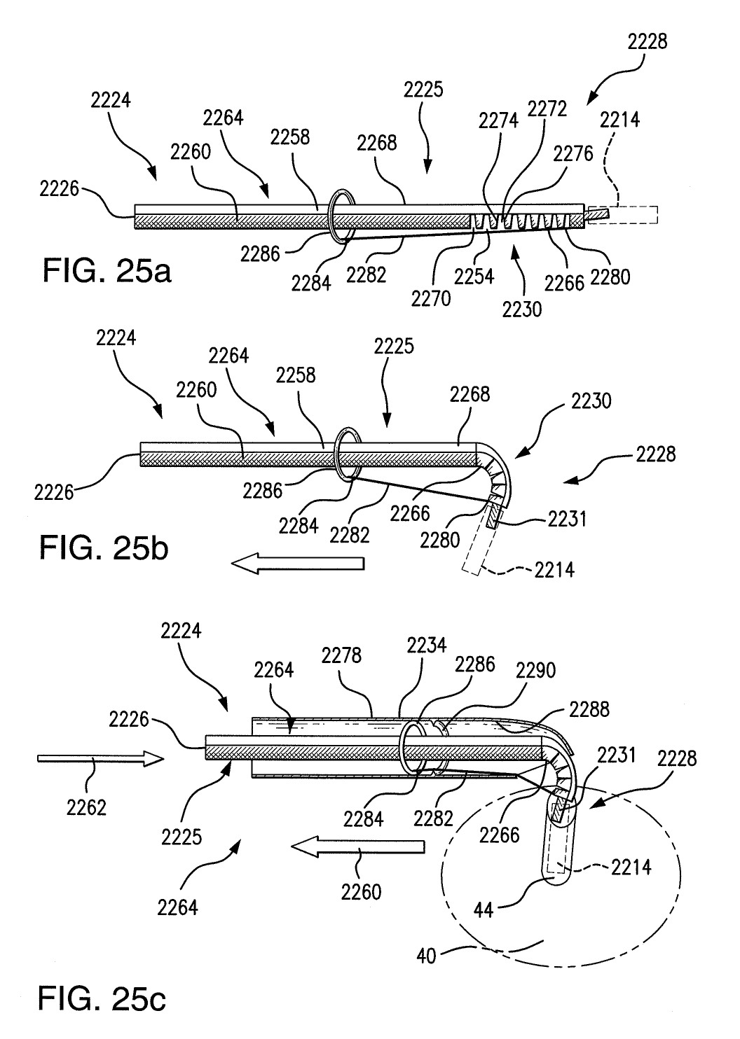

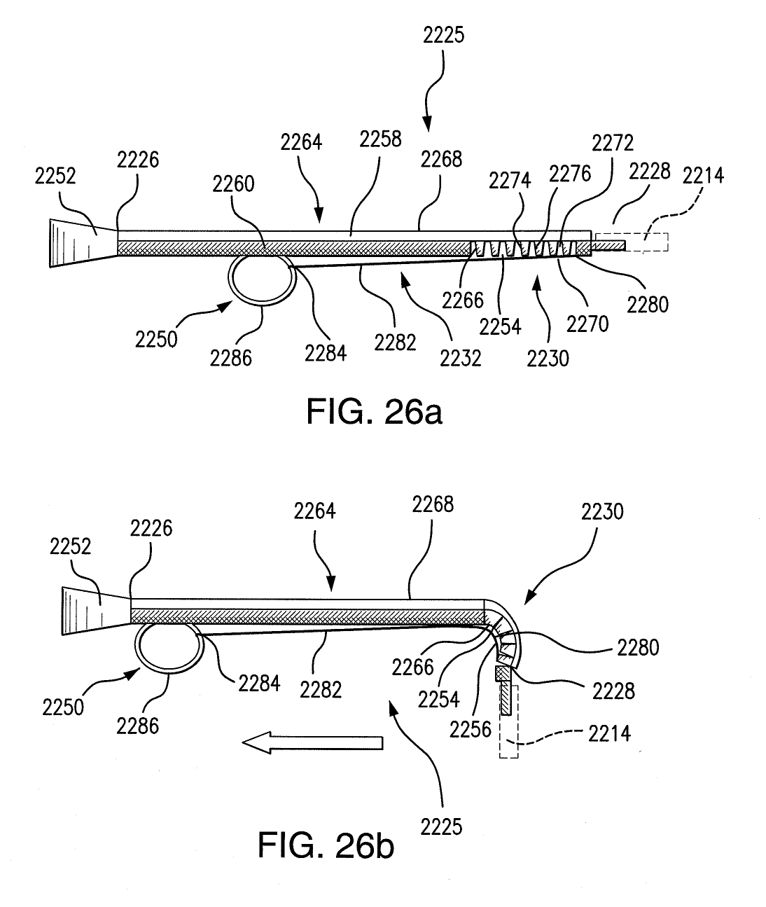

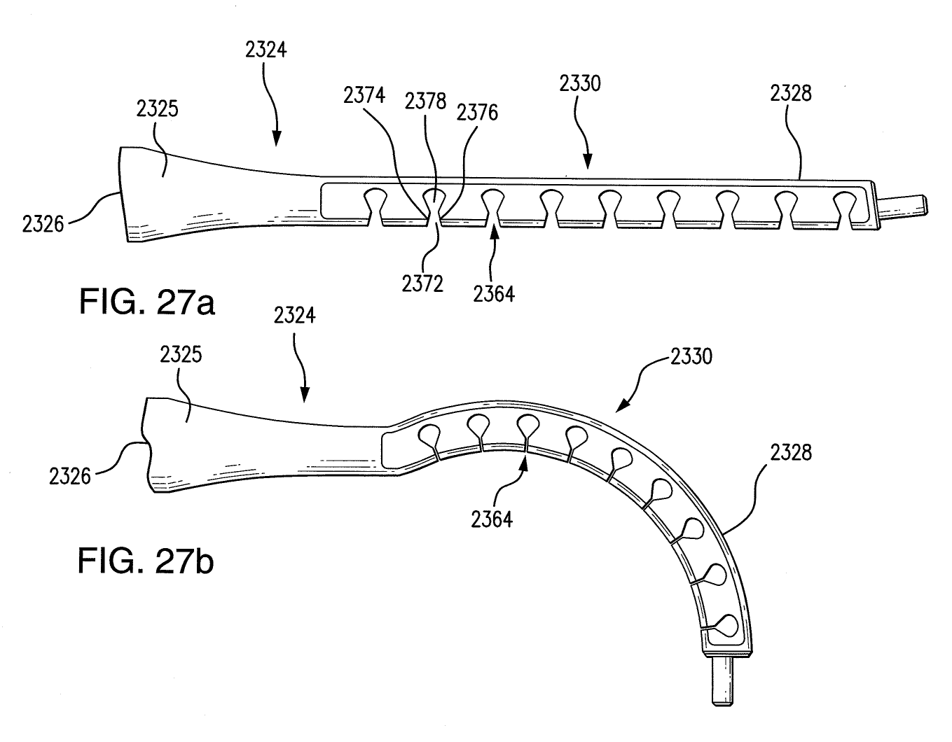

In accordance with yet another embodiment for the control rod, the flexibility of the control rod at the distal end thereof may be controlled with a construction that functions to permit bending only in one plane of motion while also limiting the total bend angle to allow for optimal insertion of the anchor. In particular, and with reference to FIGS. 24 to 33, the control rod is an elongated member extending from having a proximal end and a distal end. The control rod tapers down in thickness as it extends from the proximal end to the distal end thereof. It particular, the control rod exhibits a stepped reduction in thickness as it approaches the distal end thereof where the control rod is secured to the anchor. The distal end is therefore provided with a reduced diameter, flexible section permitting bending thereof to accommodate changes in the orientation of the anchor secured thereto.

The flexible section is composed of a single piece of flexible material capable of bending as the control rod is moved within the insertion device. In accordance with a preferred embodiment, the flexible section is composed of a flexible or malleable material such as a spring, spring like materials or Nitinol, plastic, thermoplastic (for example: PEEK®) and other machinable or moldable materials, although it is appreciated other materials offering similar flexibility and biocompatibility characteristics could certainly be used. The flexible section includes a first end and a second end. The first end of the flexible section is coupled to the distal tip of the control rod and the second end of the flexible section is coupled to the thickened relatively rigid proximal portion of the control rod. The flexible section includes a substantially smooth outer surface along one side thereof and a notched or gapped surface along the opposite side thereof. The notched surface functions to reduce the strength of the flexible member in controlled manner along the length of the flexible section by facilitating bending by minimizing the articulation force required for movement thereof.

In accordance with a preferred embodiment, the notches are shaped to limit the extent to which the flexible section is permitted to flex. In particular, each of the notches include a outwardly tapered section leading to the outer surface of the flexible section and an enlarged central recess spaced from the outer surface of flexible section when view along a plane symmetrically bisecting the control rod along the longitudinal axis thereof. The outwardly tapered section is defined by first and second opposed walls that, when viewed along the plane symmetrically bisecting the control rod along the longitudinal axis thereof, move closer together as they extend from the outer surface of the control rod toward the enlarged central recess. In this way, and when the flexible section reaches the desired extent of its flexible motion, the first and second opposed walls come into contact preventing further movement of the flexible section of the control rod. This contact further improves the load characteristics of the control rod as the anchor is seating within the bone allowing for improved impaction of the anchor while maintaining the flexibility to active the non-linear insertion. With regard to the enlarged central recesses, they function to reduce the strength of the control rod in a manner permitting the desired flex characteristics.

As discussed above, the invention of a non-linear anchor, adaption of the anchor rod junction, flexible or hinged control rod, angled guides and flexible reamer comprise a unique method of non-linear or divergent application of a suture anchor that allows for surgical repairs in anatomically confined spaces. Referring to the various figures and the embodiments disclosed therein, the present invention provides an anchor, a delivery assembly for inserting the anchor, and a method for attaching sutures to body tissue, such as bone or cartilage to repair injuries to tendons, ligaments, cartilage and other connecting structures in the body. The anchor is shaped and dimensioned for application in divergent or differing angles than the angle of the anatomically required approach. As a result, the anchor aids in improving the surgeon's ability to better approximate the open procedure endoscopically. In some cases, use of the invention presented may represent a significant improvement over traditional open techniques.

The anchor is applied in a non-linear, non-co-linear or a divergent angle to the insertion angle required to approach the anatomic structure as dictated by the confines of the local anatomy. The control rod is applicable to, but is not limited to, rotator cuff repairs, ligament repairs in the shoulder, repair of humeral avulsions of the geno-humeral ligaments and anterior cruciate ligament or knee ligament repair or reconstruction. It is appreciated that the anchor is fabricated or produced from a number of materials including, but not limited to, metals, plastic, polymers, bio-absorbable, biologic, or materials that are mechanically compatible with the flexibility required. Furthermore, and as will be appreciated based upon the following disclosure, the present invention allows for newer procedures to be developed that have not been attempted in the past by virtue of the inability to access anatomic locations in a linear approach.

More particularly, and with reference to the embodiment disclosed in FIGS. 24(a) & 24(b), 25(a)-25(c), and 26(a) & 26(b), the control rod 2224 is composed of an elongated rod body 2225. The elongated rod body 2225 includes a flexible section 2230 at the distal end 2228 thereof that is weakened by the removal of material or the formation of cuts in the control rod 2224. The removal of material and/or cuts results in the formation of gaps, notches or slits in the flexible section 2230 of the rod body 2225 of the control rod 2224. This construction provides for the control of the angle of insertion, control of the angle of curvature of the flexible section 2230, and rotational control of the insertion angle and the anchor itself. In this way, the flexible section 2230 of rod body 2225 of the control rod 2224 can be constructed to control the minimum and maximum angles of defection of the anchor 2214 from the initial direction of the control rod 2224. To aid in the control of the control rod 2224 and hence anchor insertion, the rod body 2225 of the control rod 2224 is made with a flexible section 2230 that has gaps, notches, cuts, slits or material removed in an asymmetrical way to allow for non-linear insertion of the anchor 2214 while controlling the extent of bending, rotation and length of the flexible section 2230. The amount of material removed and the depth of the cuts can be specifically tailored to the specific application of insertion of an arthroscopic anchor for soft tissue repair.

The anchor control rod 2224 can be inserted through a standard cannula or in angle directing insertion tube that guides the anchor 2214 into the angle of the required fixation tunnel 44. The control rod 2224 is accompanied by a matching guide and flexible drill or reamer that can create a tunnel at the desired angle even when that angle is different from the insertion angle or direction of the cannula, guide and reamer.

The control rod 2224 has several unique features to enable the non-linear placement of the anchor 2214. First, the flexibility of the control rod 2224 in the area of the flexible section 2230 is directionally controlled by the physical asymmetric configuration of the material making up the control rod 2224 in the area of the flexible section 2230. Furthermore, the gaps 2254 in the concave side (that is the concave side when the flexible section is flexed in accordance with the present invention) of the rod body 2225 of the control rod 2224 allow for control of the material properties to obtain the appropriate stiffness for the application of the anchor 2214. The size of each gap 2254 in the concave side is such as to control the maximum flexion angle possible in the control rod 2224 and hence anchor insertion. As the gaps 2264 close when the control rod 2224 is flexed in the area of the flexible section 2230 and the edges meet, the contact pressure of this design feature adds stiffness to the control rod 2224 as the device reaches its fully flexed position. This aids in creating the force required for proper insertion of the anchor 2214.

More particularly, and with reference to FIGS. 5(a)-(c), it is shown how a flexible drill or awl 38 can be inserted in a guide 36 to make a non-linear hole (or tunnel) 44 in the bone 40. This prepares the bone 40 for the proper positioning of the anchor 2214 to be inserted with the control rod 2224.

FIGS. 24(a) & 24(b) show, in accordance with a preferred embodiment of the present invention, the rod body 2225 of the control rod 2224 is composed of several sections. As with the various embodiments described above, the control rod 2224, that is, the rod body 2225, is an elongated member having a proximal end 2226 and a distal end 2228. The rod body 2225 of the control rod 2224 includes a substantially rigid straight section 2264 along the proximal end 2226 thereof, while the distal end 2228 is provided with the flexible section 2230 briefly discussed above. Distal to the flexible section 2230 is a distal tip 2231 shaped and dimensioned for coupling with the anchor 2214 in a manner discussed in detail above with regard to the other embodiments. The distal end 2228 of the rod body 2225 of the control rod 2224 is, therefore, provided with a flexible section 2230 permitting bending thereof to accommodate changes in the orientation of the anchor 2214 secured thereto. It is appreciated the various sections of the rod body 2225 of the control rod 2224 as it extends from the proximal end 2226 thereof to the distal end 2228 thereof may be made from the same or different materials depending upon the specific rigidity/flexibility characteristics required by the surgical procedure to be performed.

FIG. 24(a) depicts the control rod 2224 in the resting position without an anchor 2214 attached. In accordance with the embodiment disclosed herein, the control rod 2224 may be thought of as including a distal tip 2231 for holding the anchor 2214 distally, a straight section 2264 with an upper part 2258 and a lower part 2260, and a flexible section 2230 between the proximal end 2226 and the distal tip 2231. The flexible section 2230 is constructed with shaped projections 2266 extending from the upper part 2258 (the lower part 2260 along the straight section being removed). This results in a weakened area allowing for flexing thereof and the creation of the present flexible section 2230.

The projections 2266 are spaced along the upper part 2258 in a manner establishing defined gaps 2254 between the projections 2266. The shape and space between the projections 2266 is tailored to the desired bend required to insert the anchor 2214 properly. FIG. 24(b) shows the control rod 2224 in its fully bent or flexed position. The space or gaps 2254 between the material left (that is, the projections 2266) in the flexible section 2230 is eliminated and force can be transmitted at 90 degrees to the control rod's initial direction to the anchor as it is seated.

More particularly, the flexible section 2230 includes a substantially smooth outer surface 2280 along the upper surface 2258 and a gapped or notched surface 2270 along the opposite side thereof. The notched surface 2270 functions to reduce the strength of the flexible section 2230 in a controlled manner along the length of the flexible section 2230 by facilitating bending by minimizing the articulation force required for movement thereof.

In accordance with a preferred embodiment, the gaps 2254 are shaped to limit the extent to which the flexible section 2230 is permitted to flex. In particular, each of the gaps 2254 includes an outwardly tapered section 2272 leading to the outer surface of the flexible section 2230 when viewed along a plane symmetrically bisecting the rod body 2225 of the control rod 2224 along the longitudinal axis thereof. The outwardly tapered section 2272 is defined by first and second opposed walls 2274, 2276 of the respective projections 2266 that, when viewed along the plane symmetrically bisecting the rod body 2225 of the control rod 2224 along the longitudinal axis thereof, move closer together as they extend from the free end of the projections 2266 toward the upper surface 2258. In this way, and when the flexible section 2230 reaches the desired extent of its flexible motion, the first and second opposed walls 2274, 2276 come into contact preventing further movement of the flexible section 2230 of the rod body 2225 of the control rod 2224.

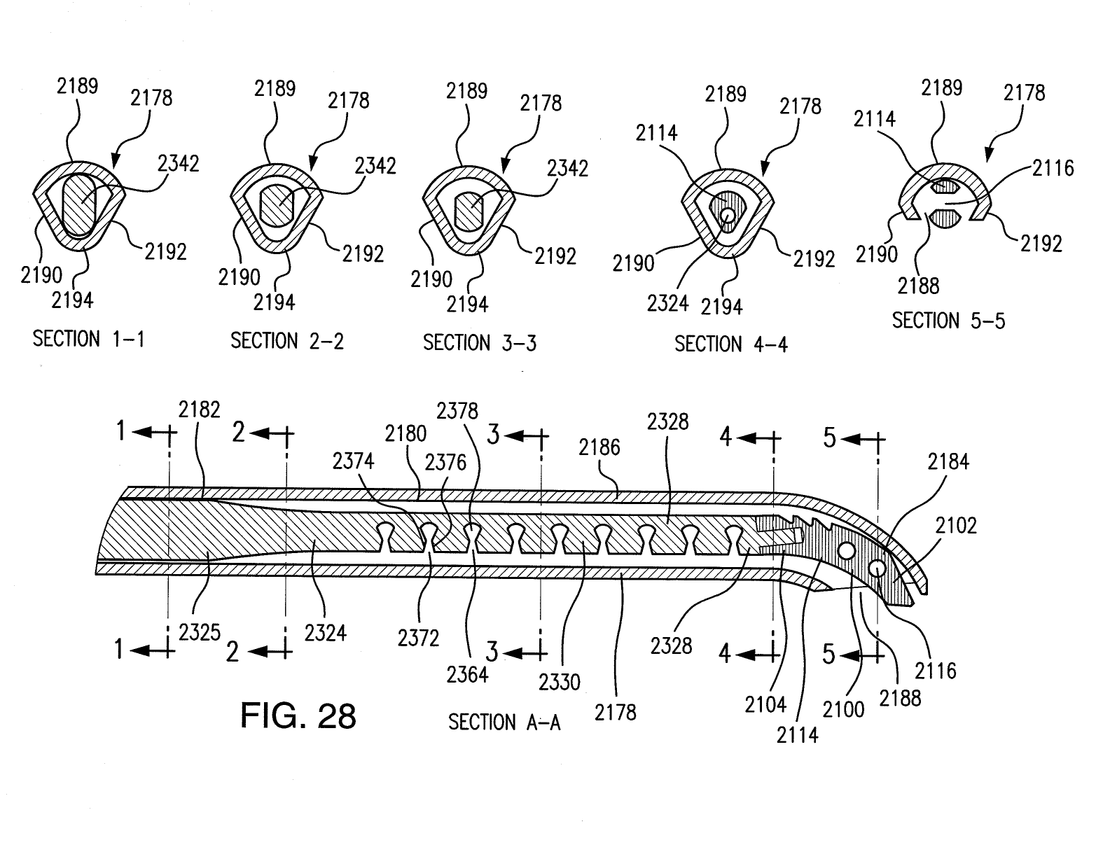

In accordance with an alternate embodiment as shown with reference to FIGS. 27(a) & 27(b), as well as FIGS. 28-33, an alternate embodiment of a control rod 2324 is disclosed. As with the prior embodiment, the control rod 2324 includes an elongated rod 2325 having a proximal end 2326 and a distal end 2328. The elongated rod body 2325 includes a flexible section 2330 at the distal end 2328 that is formed with a one piece construction with notches 2354 formed along the length of the flexible section 2330. The notches 2354 are shaped and dimensioned to limit the extent to which the flexible section 2330 is permitted to flex. In particular, each of the notches 2354 include an outwardly tapered section 2372 leading to the outer surface of the flexible section 2330 and an enlarged central recess 2378 spaced from the outer surface of flexible section 2330 when view along a plane symmetrically bisecting the rod body 2325 of the control rod 2324 along the longitudinal axis thereof. The outwardly tapered section 2372 is defined by first and second opposed walls 2374, 2376 that, when viewed along the plane symmetrically bisecting the rod body 2325 of the control rod 2324 along the longitudinal axis thereof, move closer together as they extend from the outer surface of the rod body 2325 of the control rod 2324 toward the enlarged central recess 2378. In this way, and when the flexible section 2330 reaches the desired extent of its flexible motion, the first and second opposed walls 2374, 2376 come into contact preventing further movement of the flexible section 2330 of the rod body 2325 of the control rod 2324. With regard to the enlarged central recesses 2278, they function to reduce the strength of the control rod 2324 in a manner permitting the desired flex characteristics.

Alternatively, and with reference to the embodiment of the control rod 2224 disclosed with reference to FIGS. 24(a) & 24(b), 25(a)-25(c), and 26(a) & 26(b), the control rod 2224 can also control the non-linear insertion angle without the use of excessive force on the guide 2178. In accordance with an embodiment disclosed with reference to FIGS. 25(a), 25(b) and 25(c), a first end 2280 of a tension cable 2282 is attached to the rod body 2225 of the control rod 2224 adjacent the distal tip 2231 of the control rod 2224 on the concave side thereof and a second end 2284 of the tension cable 2282 is attached to a ring (or stop) 2286 positioned along the proximal end 2226 of the rod body 2225 of the control rod 2224. This ring or stop 2286 is shaped and dimensioned to engage with the wall 2288 of the guide or inserter 2278 (see FIG. 25(c)) or function as a manual trigger 2250 (see FIGS. 26(a) and 26(b)) that would then control the bending of the control rod 2224 and hence the insertion angle of the anchor. In this way, the system allows for insertion in confined spaces at angles not previously possible.

As shown with reference to the embodiment disclosed in FIGS. 25(a), 25(b) & 25(c), a tension cable 2282 is provided on the concave side inside or along-side the control rod 2224. The first end 2280 of the tension cable 2282 is fixedly attached adjacent the distal tip 2231 of the rod body 2225 of the control rod 2224 and the second end 2284 of the tension cable 2282 is attached to the ring or stop 2286. The ring or stop 2286 is shaped and dimensioned to tension the cable 2282 and thereby control the flexion angle of the control rod 2224. This feature allows for insertion of an anchor 2214 in a non-linear direction even when contact with an outside guide 2278 is minimal or not present.

More particularly, FIG. 25(a) depicts the control rod 2224 in the resting position with an anchor 2214 attached. In this embodiment, and as with the prior embodiment, the control rod 2224, in particular, the rod body 2225, includes a proximal end 2226 and a distal end 2228. The control rod 2224 includes a distal tip 2231 for holding the anchor 2214 distally, a straight section 2264 with an upper part 2258) and a lower part 2260, and a flexible section 2230 between the proximal end 2226 and the distal tip 2231. The flexible section 2230 is constructed with shaped projections 2266 extending from the upper part 2258 (the lower part 2260 along the straight section being removed). This results in a weakened area allowing for flexing thereof and the creation of the present flexible section 2230. The projections 2266 are spaced along the upper part 2258 in a manner establishing defined gaps 2254 between the projections 2266. The shape and space between the projections 2266 is tailored to the desired bend required to insert the anchor properly. FIGS. 25(b) and 25(c) show the control rod 2224 in its fully bent or flexed position. The space or gaps 2254 between the projections 2266 of the flexible section 2230 are eliminated and force can be transmitted at 90 degrees to the control rod's initial direction to the anchor as it is seated. The flexible section 2230 includes a substantially smooth outer surface 2268 along the upper surface 2258 and a gapped or notched surface 2270 along the opposite side thereof. The notched surface 2270 functions to reduce the strength of the flexible section 2230 in controlled manner along the length of the flexible section 2230 by facilitating bending by minimizing the articulation force required for movement thereof. In accordance with a preferred embodiment and as discussed above with regard to the embodiment of FIGS. 24(a) & 24(b), the gaps 2254 are shaped to limit the extent to which the flexible section 2230 is permitted to flex.

As discussed above, the control rod 2224 includes a tension cable 2282, string or other rope like material attached to the distal tip 10 at the distal most portion of the rod body 2225 of the control rod 2224 at one end. The tension cable 2282 is attached to a ring or stop 2286 at its second end 2284 at a position more proximally along the length of the rod body 2225 of the control rod 2224. The ring 2286 may engage an inner (or outer part) of the insertion sleeve 36 shown in FIG. 1 causing the control rod 2224 to bend or flex with insertion by virtue of the tension cable's action causing flexion of the control rod 2224.

FIG. 25(b) shows the control rod 2224 in the fully bent or flexed position. The gaps 2254 between the projections 2266 on the flexible section 2230 are eliminated and force can now be transmitted at 90 degrees to the control rod's initial direction to the anchor as it is seated. The ring or stop 2286 has moved back on the control rod 12.

FIG. 25(c) shows how the stop or ring 2234 would engage the guide 2278 at the internal ridge or projection 2290. This causes the tension cable 2282 to move in the direction 2260 as the control rod 2224 is pushed into the guide 2278 in the direction 2262. This would cause the anchor 2214 to end up in the tunnel 44 in the bone 40 in the desired direction.

In accordance with another embodiment as shown with reference to FIGS. 26(a) and 26(b), the second end 2284 of the tension cable 2282 to which a ring 2286 is secured can function as trigger 2250 that can be used by the operator to curve the anchor into place without the use of an external guide. FIGS. 26(a)-(b) demonstrate another embodiment with a handle 2252 at the proximal end 2226. It shows a ring 2286 in the form of a trigger 2250 that may be operated by the surgeon. When moved in the direction 2254, the distal tip 2231 of the control rod 2224 flexes though the action of the cable system 2232 causing the tip to flex to the desired curved position 2256. Additional embodiments of this design are envisioned were the trigger and handle are gun shaped for better ergonomic handling. Further designs are envisioned were the gun shape handle and inserter guide are one unit to ensure the force required to turn the anchor in the guide to be applied against the handle and guide instead of the bone so in soft bone applications the anchor will not cut through the bone in an undesirable direction.

Although the use of a control rod is disclosed with reference to the embodiment of the control rod 2224 disclosed with reference to FIGS. 24(a) & 24(b), 25(a)-25(c), and 26(a) & 26(b), it is appreciated such controlled flexion could be applied to the embodiment of the control rod disclosed with reference to FIGS. 27(a) and 27(b).

Referring now to FIGS. 28-33 a representative embodiment with the triangular anchor 2114 disclosed in FIGS. 23(a)-23(i) and the control rod 2324 of FIGS. 27(a) & 27(b), as well as an another embodiment of the insertion device, or guide, 2178, is disclosed. The guide 2178 is also triangular in shape when view along a cross section perpendicular to a longitudinal axis thereof. As such, and as with the previously discussed embodiments of the insertion device, it is an elongated hollow member 2180 having a first end 2182 and a second end 2184. A conduit extends between the first end 2182 and the second end 2184 allowing for the passage of an anchor 2114 in accordance with the present invention. The conduit along the first end 2182 and the central portion 2186 of the insertion device 2178 is substantially straight and parallel to the longitudinal axis of the insertion device 2178. At the second end 2184 of the insertion device 2178, that is, at the outlet end of the insertion device 2178, the conduit turns such that the conduit makes a substantially 90 degree turn allowing for deployment of the anchor 2114 from the outlet 2188 at the second end 2184 of the insertion device 2178 in a direction substantially transverse to the longitudinal axis of the insertion device 2178. In accordance with a preferred embodiment, the conduit is curved in the turning section so as to guide the anchor therethrough while limiting resistance to movement.

The insertion device 2178 includes a substantially triangular shape when viewed along a cross section taken perpendicular to a longitudinal axis of the insertion device 2178. As such, the insertion device 2178 includes a top wall 2189. The top wall 2189 includes a convex arcuate shape as it extends as it extends from the first and second lateral side walls 2190, 2192 of the insertion device 2178. The first and second lateral side walls 2190, 2192 of the insertion device 2178 extend from the top wall 2189 at an acute angle such that they meet at the bottom wall 2194 of the insertion device 2178. The bottom wall 2194 has a convex arcuate shape (that is, rounded) as it extends from the first and second lateral side walls 2190, 2192 of the anchor body 2100.

As discussed above, the control rod 2124 is an elongated member having a proximal end 2126 and a distal end 2128. In contrast to the anchor 2114 and insertion device 2178, the control rod 2124 includes a substantially oblong shaped when viewed along a cross section taken perpendicular to a longitudinal axis of the insertion device 2178. The control rod 2124 tapers down in thickness as it extends from the proximal end 2126 to the distal end 2128 thereof. It particular, the control rod 2124 exhibits a stepped reduction in thickness as it approaches the distal end 2128 thereof where the control rod 2124 is secured to the anchor 2114. The distal end 2128 is therefore provided with a reduced diameter, flexible section 2130 permitting bending thereof to accommodate changes in the orientation of the anchor 2114 secured thereto. The staged diameter control rod 2124 with round cross section (so it can be unscrewed if it is desired to have a screw-in attachment to the anchor 2114) provides a better fit within insertion device 2178 proximally while allowing for a flexible section 2130 to make the turn—still enabling the ability to tap the anchor 2114 in with a small mallet because the flexible section is short and the anchor has shape to prevent twisting.

As is appreciated based upon the drawings, the anchor 2114 and control rod 2124 are sized and shaped to fit within the insertion device 2178. The provision of a substantially triangular anchor 2114 allows for enhanced control of rotation of the anchor 2114 and control rod 2124 (which is secured to the anchor) as the control rod 2124 is pushed through the insertion device 2178 for deployment of the anchor 2114. The triangular configuration also permits the creation of a wider back (that is, a bigger lateral radius arc along the top surface 2150 as the top surface 2150 extends from the first and second lateral sides 2162, 2164 of the anchor 2114) which helps stop pushing through softer bone and better turning. The triangular shape also assists in creating a space in the bone tunnel for sutures to slide better and better locking in the bone once turning to lock or flip in place forming a wedge shape against the direction of anchor pull out enhancing pull out strength of the anchor design. After ease of insertion, anchor pull out test results is considered one of the most important surgeon anchor choice decision attributes.

In addition, the non-round cross sectional profile of the insertion device 2178 allows the anchor 2114 to fit better therein and control anchor movement down the insertion device 2178.

It is appreciated the insertion device 2178 disclosed above with reference to FIGS. 28-33 may be manufactured with a circular cross section instead of a triangular cross section.

Referring now to FIG. 29, this is a cross sectional schematic view showing deployment of the anchor 2114 in accordance with embodiment disclosed with reference to FIGS. 23(a)-23(i) using the control rod 2324 of FIGS. 27(a) & 27(b).

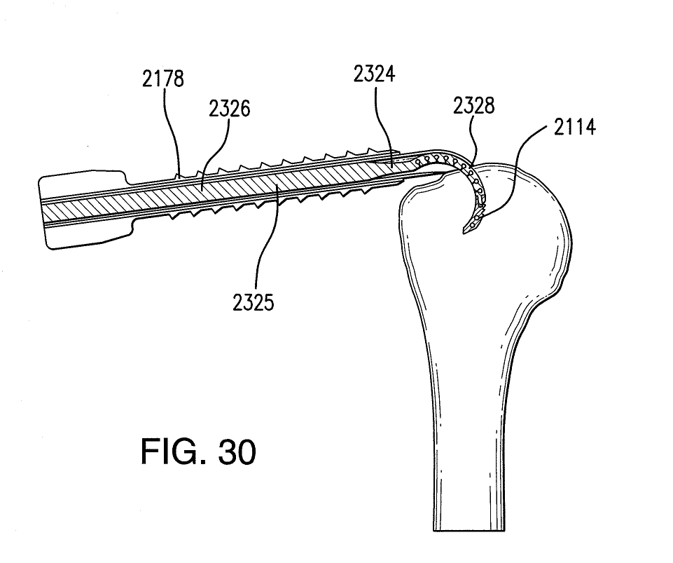

FIGS. 30, 31, 32 and 33 are cross sectional views showing deployment of the anchor 2114 in accordance with embodiment disclosed with reference to FIGS. 23(a)-23(i) using the control rod 234 of FIGS. 27(a) & 27(b).

It is appreciated the flexible section can be made with material removed in a number of configurations or shapes to best control the flexibility of the control rod on order to match the properties of the material the anchor is inserted into. In our case this may mean different rods for normal young hard bone, average bone strength, or osteoporotic softer bone.

While the preferred embodiments have been shown and described, it will be understood that there is no intent to limit the invention by such disclosure, but rather, is intended to cover all modifications and alternate constructions falling within the spirit and scope of the invention.

1. A suture anchor and delivery assembly, comprising:
an elongated, non-linear anchor body with a first end and a second end, at least one hole is formed in the anchor body for the passage of a suture therethrough, the anchor body including an attachment site at a second end thereof;
a control rod shaped and dimensioned for attachment to the attachment site of the anchor body, the control rod includes a distal end constructed of a flexible materials allowing for displacement of the control rod; and
a guide for the insertion of the anchor.

2. The suture anchor and delivery assembly according to claim 1, wherein the control rod has a section of a flexible material shaped to permit bending only in one desired plane of motion while limiting the total bend angle to allow for optimal insertion of the anchor.

3. The suture anchor and a delivery assembly according to claim 1, wherein the anchor and the control rod are coupled via a ball joint.

4. The suture anchor and a delivery assembly according to claim 1, wherein the guide is an elongated hollow member having a first end and a second end with a conduit extending between the first end and the second end allowing for the passage of the anchor and the guide includes an outlet end at the second end of the guide wherein the conduit turns allowing for deployment of the anchor at the second end of the guide in a direction substantially transverse to the longitudinal axis of the guide.

5. The suture anchor and a delivery assembly according to claim 1, wherein the anchor includes a cannulation down a center of the anchor body.

6. The suture anchor and a delivery assembly according to claim 1, wherein the anchor includes a first anchor section and a second anchor section, the first anchor section and the second anchor section being coupled with hinges permitting relative movement.

7. The suture anchor and a delivery assembly according to claim 1, wherein anchor is triangular in shape when viewed along a cross section taken perpendicular to a longitudinal axis of the anchor body, and the guide is also triangular in shape when view along a cross section perpendicular to a longitudinal axis thereof.

8. The suture anchor and a delivery assembly according to claim 7, wherein the guide includes a top wall extending between first and second lateral side walls of the guide, the first and second lateral side walls of the guide extend from the top wall at an acute angle such that they meet at a bottom wall of the guide.

9. The suture anchor and a delivery assembly according to claim 1, wherein the control rod includes a substantially rigid straight section along a proximal end thereof and a flexible section at a distal end, the flexible section being weakened by removal of material or the formation of cuts in the flexible section of the control rod.

10. The suture anchor and a delivery assembly according to claim 9, wherein the flexible section includes gaps allowing for control of material properties to obtain appropriate stiffness for application of the anchor, wherein the size of each gap is such as to control maximum flexion angle possible in the control rod.

11. The suture anchor and a delivery assembly according to claim 10, wherein each of the gaps includes an outwardly tapered section leading to an outer surface of the flexible section when viewed along a plane symmetrically bisecting the control rod along a longitudinal axis thereof, the outwardly tapered section is defined by first and second opposed walls of the respective projections that, when viewed along the plane symmetrically bisecting the control rod along the longitudinal axis thereof, move closer together as they extend from a free end of the projections toward an upper surface of the control rod such that when the flexible section reaches a desired extent of its flexible motion, the first and second opposed walls come into contact preventing further movement of the flexible section of the control rod.

12. The suture anchor and a delivery assembly according to claim 9, wherein the flexible section is formed with notches formed along a length of the flexible section.

13. The suture anchor and a delivery assembly according to claim 12, wherein each of the notches include an outwardly tapered section leading to an outer surface of the flexible section and an enlarged central recess spaced from the outer surface of the flexible section when view along a plane symmetrically bisecting the control rod along the longitudinal axis thereof.

14. The suture anchor and a delivery assembly according to claim 9, wherein the control rod includes tension cable controlling flexing of the flexible section.

15. The suture anchor and a delivery assembly according to claim 14, wherein the first end of the tension cable is attached to the control rod adjacent a distal tip of the control rod and a second end of the tension cable is attached to the proximal end of the control rod.

16. The suture anchor and a delivery assembly according to claim 15, wherein the second end of the tension cable is attached a stop shaped and dimensioned to engage with a wall of the inserter.

17. The suture anchor and a delivery assembly according to claim 15, wherein the second end of the tension cable is attached to a stop functioning as a manual trigger controlling the bending of the control rod.

18. A method for attaching sutures to body tissue, such as bone or cartilage to repair injuries to tendons, ligaments, cartilage and other connecting structures in the body by application of a bone anchor in a non-linear, non-co-linear or a divergent angle to the insertion angle required to approach the anatomic structure as dictated by the confines of the local anatomy, comprising:
forming a tunnel within an anatomical site;
positioning a guide adjacent the tunnel, the guide including an angled outlet end having an opening transverse to the longitudinal axis of the guide.
passing a bone anchor through the guide to, and out of the outlet end of the guide;
positioning the anchor with the tunnel; and
seating and locking the anchor in the tunnel.

19. The method according to claim 18, wherein the an inner surface of the guide at the outlet end is provided with a partial spherical shape shaped and dimensioned to direct the anchor to the outlet at the outlet end of the guide and into the tunnel.

20. The method according to claim 18, wherein the anchor is coupled to a control rod which is composed of a proximal rigid core and distal flexible segment.

21. The method according to claim 18, wherein the anchor is wherein the anchor is non-linear.

22. The method according to claim 18, wherein the anchor is curved.

23. A suture anchor for use in non-linear, non-co-linear or divergent angle deployment, comprising:
an anchor body including a first end and a second end, at least one hole is formed in the anchor body for the passage of a suture therethrough, the anchor body including an attachment site at a second end thereof;
wherein the anchor body is triangular in shape when viewed along a cross section taken perpendicular to a longitudinal axis of the anchor body.

24. The suture anchor and a delivery assembly according to claim 23, wherein the anchor includes a top surface extending from a first end to a second end of the anchor body, first and second lateral sides extend from the top surface at an acute angle such that they meet at a bottom surface of the anchor body.

25. The suture anchor and a delivery assembly according to claim 24, wherein the anchor includes a suture hole that extends through the anchor body from the first lateral side thereof to the second lateral side thereof at a position between the top surface and the bottom surface.

26. A control rod for a suture anchor and delivery assembly, comprising:
an elongated rod body shaped and dimensioned for attachment to an attachment site of the anchor body, the rod body includes a distal end constructed of a flexible materials allowing for displacement of the control rod;
the rod body further including a substantially rigid straight section along a proximal end thereof and a flexible section at a distal end, the flexible section being weakened by removal of material or the formation of cuts in the flexible section of the rod body.

27. The control rod according to claim 26, wherein the flexible section includes gaps allowing for control of material properties to obtain appropriate stiffness for application of the anchor, wherein the size of each gap is such as to control maximum flexion angle possible in the rod body.

28. The control rod according to claim 27, wherein each of the gaps includes an outwardly tapered section leading to an outer surface of the flexible section when viewed along a plane symmetrically bisecting the rod body along a longitudinal axis thereof, the outwardly tapered section is defined by first and second opposed walls of the respective projections that, when viewed along the plane symmetrically bisecting the rod body along the longitudinal axis thereof, move closer together as they extend from a free end of the projections toward an upper surface of the rod body such that when the flexible section reaches a desired extent of its flexible motion, the first and second opposed walls come into contact preventing further movement of the flexible section of the rod body.

29. The control rod according to claim 26, wherein the flexible section is formed with notches formed along a length of the flexible section.

30. The control rod according to claim 29, wherein each of the notches include an outwardly tapered section leading to an outer surface of the flexible section and an enlarged central recess spaced from the outer surface of the flexible section when view along a plane symmetrically bisecting the rod body along the longitudinal axis thereof.

31. The control rod according to claim 26, further including tension cable controlling flexing of the flexible section.

32. The control rod according to claim 31, wherein the first end of the tension cable is attached to the rod body adjacent a distal tip of the rod body and a second end of the tension cable is attached to the proximal end of the rod body.

33. The control rod according to claim 32, wherein the second end of the tension cable is attached a stop shaped and dimensioned to engage with a wall of a guide.

34. The control rod according to claim 32, wherein the second end of the tension cable is attached to a stop functioning as a manual trigger controlling the bending of the rod body.
